(19) United States
(12) Patent Application Publication
(10) Pub. No.: US 2018/0133050 A1
(43) Pub. Date: May 17, 2018

(54) ICE THERAPY DEVICE

(71) Applicant: Deborah Manning, Moorpark, CA (US)

(72) Inventor: Deborah Manning, Moorpark, CA (US)

(21) Appl. No.: 15/809,740

(22) Filed: Nov. 10, 2017

Related U.S. Application Data
(60) Provisional application No. 62/420,741, filed on Nov. 11, 2016.

(51) Int. Cl. A61F 7/02

(52) U.S. Cl. CPC A61F 7/02; A61F 2007/0042

(57) ABSTRACT

An ice therapy device adapted to provide ice therapy to a portion of a body of an animal or an individual. The ice therapy device comprising at least a first section, at least a second section, at least one joint, and at least one seam, wherein the at least one joint couples the first section and second section together. The first and second sections comprising a pouch configured to house ice cells. The ice therapy device further comprising at least one strap, such that the at least one strap is configured to be removably coupled to the outer surface of the ice therapy device. The at least one seam is adjacent the at least one joint and extends towards an outer edge of the first and second sections, and provides a separation between the first and second section, such that the ice therapy device allows for mobility.

### RELATED APPLICATION

This application claims the benefit of priority under 35 USC 119(e) to U.S. Provisional Application Ser. No. 62/420,741 to Deborah Manning, which was filed on Nov. 11, 2016. U.S. Provisional Application Ser. No. 62/420,741 including its drawings, schematics, diagrams, and written description, is hereby incorporated in its entirety by reference.

### BACKGROUND

### Field

This disclosure relates to an ice therapy device adapted to provide ice therapy. More specifically, the disclosure is directed to a garment that is configured to provide ice therapy to at least a portion of body of an animal or an individual.

### Description of the Related Art

Cryotherapy is the application of ice or cold to an injured area for a therapeutic effect. Cold therapy is often the immediate first aid applied as soon as possible for many sports injuries helping to reduce pain, swelling and inflammation. Cold therapy is used to manage the magnitude of the inflammatory process, blood flow, initial swelling, secondary injury and pain. The use of ice packs is widespread because of their effectiveness, convenience, low cost, and ease of transportation.

Typically, an ice pack is placed over an injured area and is intended to absorb heat of a closed traumatic or edematous injury by using conduction to transfer thermal energy. The physiologic effects of cold application include immediate vasoconstriction with reflexive vasodilation, decreased local metabolism and enzymatic activity, and decreased oxygen demand. Cold decreases muscle spindle fiber activity and slows nerve conduction velocity, therefore it is often used to decrease spasticity and muscle guarding. It is commonly used to alleviate the pain of minor injuries, as well as decrease muscle soreness. The use of ice packs in treatment decreases the blood flow most rapidly at the beginning of the cooling period. Ice is a very popular modality for treatment in injuries and muscle repair following any activity.

Cold therapy is typically used to reduce pain and swelling from a sports or activity injury to soft tissues. The therapy is useful for sprains, strains, pulled muscles and/or ligaments. The use of cryotherapy in acute injury management lowers the temperature of the injured tissue, which reduces the tissue's metabolic rate and helps the tissue to survive the period following the injury.

Cold wraps can be used to apply the ice or cold pack to the desired body part. Many of the wraps available use adjustable elastic straps to secure the wrap to the body part. Some wraps are used to hold an ice pack at the desired area and require the wrap to be wrapped around the desired area, i.e. torso, arm, or leg, and these wraps require that the wrap be wrapped around the desired area in order to hold the ice pack in place. Wrapping an ice pack around a particular body part can require an excessive amount of wrapping in order to hold the ice pack at the desired area due to gravity and/or the desired area being at a point of the body that does not easily allow it to be wrapped. Securing an ice pack to certain body parts can be difficult and could lead to discomfort due to the bulk of the ice pack. People that have ice packs wrapped on their body, arms, and/or legs, are typically in a resting position, such as sitting or lying down, and are not usually performing any sort of physical activity and/or motion.

Cold therapy is not limited to humans. Cold therapy is also used on animals, such as but not limited to horses, dogs, sporting animals, and the like. Appling cold therapy to a horse's leg after a workout is known, but can be harmful to the horse if not done properly. When a horse is working hard, the capillaries that extend into the muscles, tendons and ligaments expand to bring in needed blood. After the completion of the workout, the increased blood flow can continue and will bring in unneeded fluid that contains mediators and enzymes associated with inflammation. Pooling fluids will stretch tissues, making them less elastic over time, and a loss of elasticity in tissues cannot be regained, which can cause the horse to be prone to fluid accumulation. Cold therapy after a workout assists in reducing the increased blood flow, and returning the horse back to a baseline circulation.

Applying an ice pack to a horse's leg can be difficult, especially if an ice pack is held onto the horse's leg using a bandage or plastic wrap. Conventional wrapping methods used by humans might not be as effective on horses and other animals. As such, wraps and/or boots can be used to apply cold therapy to a horse's leg. However, some conventional boots and/or wraps can cause discomfort to the horse and/or do not apply uniform coldness. For example, some boots have pockets that require ice to be inserted into the pocket before applying onto the horse's leg. These boots can lead to ice coming out of the pocket in certain situations, such as if the horse were to walk or move about while wearing the boot, and could cause the boot to slip off the leg, potentially tripping the horse. The potential for injury to the horse could be disastrous. Also, the ice in the pocket could be shaped such that when the boot is applied to the horse, the ice could cause discomfort against the horse's leg, or the shape of the ice does not substantially conform to the shape of the horse's leg. In such instances, the ice may need to be crushed, but crushing the ice requires an ice crusher and access to an ice crusher may not always be available. Pre-crushing ice ahead of time could be done, but storing the crushed ice for extended periods of time can be difficult because the crushed ice melts faster than an ice cube, and because crushed ice tends to join back together. Furthermore, the boots with pockets are not permeable to air and/or water, or else the boot would leak water as the ice within the pocket melts. As such, the boot could trap heat within the boot and the leg, such that the horse's leg could be hot after the boot is removed. At the end of the cold therapy session, the ice has melted and is discarded and is not reusable, such that a new supply of ice is required for subsequent cold therapy sessions. The supply of ice could be costly and difficult to keep frozen while transporting to an off-site location.

Other boots or wraps require that the horse be stationary and do not allow the horse to walk while wearing the boot or wrap. Some boots can be stiff and inflexible, whereas other boots use cold water to apply cold therapy, instead of ice, wherein a pump circulates the cold water through the boots in order to apply the cold therapy to the horse's leg. Some wraps cover only a portion of the horse's leg, such as the cannon portion or the gaskin portion of the leg. These wraps may allow the horse to walk, but they do not provide uniform coldness to the leg. Also, the leg, as a whole, does not experience a consistent cold therapy session because some parts of the leg are not wrapped while other parts are. Some wraps do allow for the wrap to cover a substantial portion of the leg, but also limit mobility of the horse. Some wraps do not use ice but instead use cold water, wherein the cold water is applied to the wrap and then the wrap is applied onto the leg of the horse.

The wraps and/or boots that are typically used for horses can be molded to the shape of the horse's leg. As such, these wraps and/or boots are limited to being used on horses, and are not intended to be also used on humans.

The disclosure is an ice therapy device adapted to provide ice therapy to a portion of a body of an animal or an individual. For example, ice therapy device can be configured to be universal, such that the ice therapy device can be used by either an animal or a person. The present disclosure addresses these needs and provides further related advantages.

### SUMMARY

The disclosure disclosed herein provides various aspects of an ice therapy device that is cost effective, reusable, and easy to apply to a human and/or animal in order to provide cold therapy to the human and/or animal. The different aspects comprise elements to allow the ice therapy device to be universal, such that the device can be used to provide cold therapy to either a person or an animal. The ice therapy device allows for the person or the animal wearing the device to have some range of motion during the cold therapy session, such that the wearer does not have to be stationary while wearing the device.

In one aspect, as broadly described herein, an ice therapy device comprises at least a first section, at least a second section, at least one joint, and at least one seam, wherein the at least one joint couples the first section and second section together. The first section comprises an inner surface and an outer surface that are coupled together to form a pouch, wherein the pouch is configured to house ice cells. The second section also comprises an inner surface and an outer surface coupled together to form a pouch that houses ice cells. Each of the first and second sections further comprise at least one strap, such that the at least one strap is configured to be removably coupled to the outer surface of the first and second sections. The at least one seam is adjacent the at least one joint and extends towards an outer edge of the first and second sections. The at least one seam provides a separation between the first and second section, such that the ice therapy device allows for mobility while wearing the ice therapy device.

In another aspect, an ice therapy device configured for placement around a limb of an animal or a person comprising a first section, a second section, at least one joint, an opening, and at least one seam, wherein part of the first and second sections are coupled to each other by the joint, such that the opening is interposed between the first and second sections. The first and second sections comprise an inner surface, an outer surface, and a pouch interposed between the inner and outer surfaces, wherein the pouch receives ice cells. The ice therapy device further comprises at least one strap coupled to each of the first and second sections, wherein the at least one strap of the first and second sections are configured to be removably coupled to the outer surface in order to wrap the ice therapy device to the limb of the person or the animal. The opening can be arranged to be placed proximate a limb joint of the animal or person such that the ice therapy device allows for the limb joint to bend while wrapped around the limb. The at least one seam is proximate the joint and opening and extends towards an outer edge of the ice therapy device to further allow for limb mobility while the device is wrapped around the limb.

In another aspect, an ice therapy device configured for placement around one or more body parts of an animal or a person comprising a first section and a second section at least partially coupled to each other, ice cells within each of the first section and the second section, at least one seam between the first and second sections such that the ice therapy device can accommodate different body parts. The ice therapy device further comprising straps configured to be removably coupled to an outer surface of the first and/or second sections to secure the ice therapy device to the one or more body parts.

These and other aspects and advantages of the disclosure will become apparent from the following description and accompanying drawings which illustrate, by way of example, features of the disclosure.

### BRIEF DESCRIPTION OF THE DRAWINGS

FIG. 1 is a perspective view of an ice therapy device according to an aspect of the disclosure.

FIG. 2 is another perspective view of the ice therapy device of FIG. 1.

FIG. 3 is another perspective view of the ice therapy device of FIG. 1.

FIG. 4 is a side view of the ice therapy device of FIG. 1.

FIG. 5 is another side view of the ice therapy device of FIG. 1.

FIG. 6 is a perspective view of an ice therapy device according to an aspect of the disclosure.

FIG. 7 is a perspective view of the ice therapy device of FIG. 6.

FIG. 8 is a side view of the ice therapy device of FIG. 6.

FIG. 9 is another side view of the ice therapy device of FIG. 6.

FIG. 10 is a perspective view of the ice therapy device of FIG. 6.

FIG. 11 is a perspective view of an ice therapy device according to an aspect of the disclosure.

FIG. 12 is another perspective view of the ice therapy device of FIG. 11.

FIG. 13 is another perspective view of the ice therapy device of FIG. 11.

FIG. 14 is a side view of the ice therapy device of FIG. 11.

FIG. 15 is another side view of the ice therapy device of FIG. 11.

FIG. 16 is a perspective view of an ice therapy device according to an aspect of the disclosure.

FIG. 17 is another perspective view of the ice therapy device of FIG. 16.

FIG. 18 is another perspective view of the ice therapy device of FIG. 16.

FIG. 19 is a side view of the ice therapy device of FIG. 16.

FIG. 20 is another side view of the ice therapy device of FIG. 16.

FIG. 21 is a perspective view of an ice therapy device according to an aspect of the disclosure.

FIG. 22 is another perspective view of the ice therapy device of FIG. 21.

FIG. 23 is a front view of the ice therapy device of FIG. 21.

FIG. 24 is a rear view of the ice therapy device of FIG. 21.

FIG. 25 is a side view of the ice therapy device of FIG. 21.

FIG. 26 is another side view of the ice therapy device of FIG. 21.

FIG. 27 is another side view of the ice therapy device of FIG. 21.

FIG. 28 is another side view of the ice therapy device of FIG. 21.

FIG. 29 is a perspective view of an ice therapy device according to an aspect of the disclosure.

FIG. 30 is another perspective view of the ice therapy device of FIG. 29.

FIG. 31 is a side view of an ice therapy device of FIG. 29.

FIG. 32 is another side view of the ice therapy device of FIG. 29.

FIG. 33 is an overhead view of the ice therapy device of FIG. 29.

FIG. 34 is a side view the ice therapy device of FIG. 29.

FIG. 35 is a front view of the ice therapy device of FIG. 29.

FIG. 36 is a rear view of the ice therapy device of FIG. 29.

FIG. 37 is a perspective view an ice therapy device according to an aspect of the disclosure.

FIG. 38 is a perspective view of the ice therapy device of FIG. 37.

FIG. 39 is a side view of the ice therapy device of FIG. 37.

FIG. 40 is another side view of the ice therapy device of FIG. 37.

FIG. 41 is a front view of the ice therapy device of FIG. 37.

FIG. 42 is a front view of the ice therapy device of FIG. 37.

FIG. 43 is a rear view of the ice therapy device of FIG. 37.

FIG. 44 is a side view of the ice therapy device of FIG. 37.

FIG. 45 is a perspective view of an ice therapy device according to an aspect of the disclosure.

FIG. 46 is a perspective view of the exterior of the ice therapy device of FIG. 45.

FIG. 47 is a front view of the ice therapy device of FIG. 45.

FIG. 48 is a side view of the ice therapy device of FIG. 47.

FIG. 49 is another side view of the ice therapy device of FIG. 47.

### DETAILED DESCRIPTION

The disclosure described herein is directed to different aspects of an ice therapy device that in some aspects is configured to provide cold therapy to an animal or a person. The ice therapy device can comprise many different materials and can be used to provide cold therapy to many different body parts of a person such as, but not limited to, arms, legs, shoulders, and torso. The ice therapy device can also be used to provide cold therapy to many different body parts of a horse such as, but not limited to, legs, back, neck, stifle, shoulder. The ice therapy device according to the disclosure can be arranged in many different ways with many different components, and is generally arranged to provide an ice therapy device that can be used by either animals or humans and is configured to allow for mobility during the ice therapy treatment.

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent, however, to those skilled in the art that these concepts may be practiced without these specific details. In some instances, well-known structures and components are shown in block diagram form in order to avoid obscuring such concepts. As described herein, the use of the term “and/or” is intended to represent an “inclusive OR”, and the use of the term “or” is intended to represent an “exclusive OR”.

In one aspect, as broadly described herein, an ice therapy device comprises at least a first section, at least a second section, at least one joint, and at least one seam, wherein the at least one joint couples the first section and second section together. The first section comprises an inner surface and an outer surface that are coupled together to form a pouch, wherein the pouch is configured to house ice cells. The second section also comprises an inner surface and an outer surface coupled together to form a pouch that houses ice cells. Each of the first and second sections further comprise at least one strap, such that the at least one strap is configured to be removably coupled to the outer surface of the first and second sections. The at least one seam is adjacent the at least one joint and extends towards an outer edge of the first and second sections. The at least one seam provides a separation between the first and second section, such that the ice therapy device allows for mobility while wearing the ice therapy device. The at least one joint and seam work together to allow for a substantially unencumbered range of motion during the ice therapy treatment, which provides comfort and ease of use during the treatment.

The ice therapy device can provide a number of additional advantages beyond those mentioned above. For example, the ice cells can be affixed within the pouch of either the first or second sections, such that the pouch does not need to be filled with ice for cold therapy sessions. Additionally, the ice cells can be reusable such that the ice cells can be refrozen after a cold therapy session, which eliminates the need for ice. The ice cells allow the ice therapy device to easily conform to the body part to which the ice therapy device is applied, which provides a more uniform distribution of coldness. Also, the ice therapy device can be air and/or water permeable, such that the ice therapy device does not trap in heat between the body part and the ice therapy device. Conventional devices that use ice have pockets that are filled with ice, but the pockets are not permeable to air and/or water which can lead to heat being trapped between the body part and the device. The ice therapy device of the disclosure overcomes such instances, and allows heat to dissipate when using the ice therapy device.

Some aspects of the ice therapy device according to the disclosure can be used to provide cold therapy to an animal or a person. However, the disclosure is not intended to be limited to such aspects. The ice therapy device can be arranged to minimize pressure on a joint or injured area of the animal or person. For example, the ice therapy device can comprise one or more openings 115, wherein the one or more openings 115 accommodate body parts of the animal or person such that the animal or person is not physically restrained by the ice therapy device. The ice therapy device can be strapped to the animal or person, and allows the animal or person to have a substantial full range of motion.

The disclosure is described herein with reference to certain aspects, but it is understood that the disclosure can be embodied in many different forms and should not be construed as limited to the aspects set forth herein. In particular, the disclosure is described below in regards to an ice therapy device to provide cold therapy to an animal or a person, but it is understood that the disclosure can be used for many other applications in many different settings. The components of the ice therapy device can have different shapes and sizes beyond those shown in the figures or discussed herein.

Although the terms first, second, etc. may be used herein to describe various elements or components, these elements or components should not be limited by these terms. These terms are only used to distinguish one element or component from another. Thus, a first element discussed herein could be termed a second element without departing from the teachings of the disclosure. It is understood that actual ice therapy devices embodying the disclosure can be arranged in many different ways with many more features and elements beyond what is shown in the figures.

It is to be understood that when an element or component is referred to as being “on” another element or component, it can be directly on the other element or intervening elements may also be present. Furthermore, relative terms such as “between”, “within”, “below”, and similar terms, may be used herein to describe a relationship of one element or component to another. It is understood that these terms are intended to encompass different orientations of the device in addition to the orientation depicted in the figures.

Aspects of the disclosure are described herein with reference to illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region of a device and are not intended to limit the scope of the disclosure.

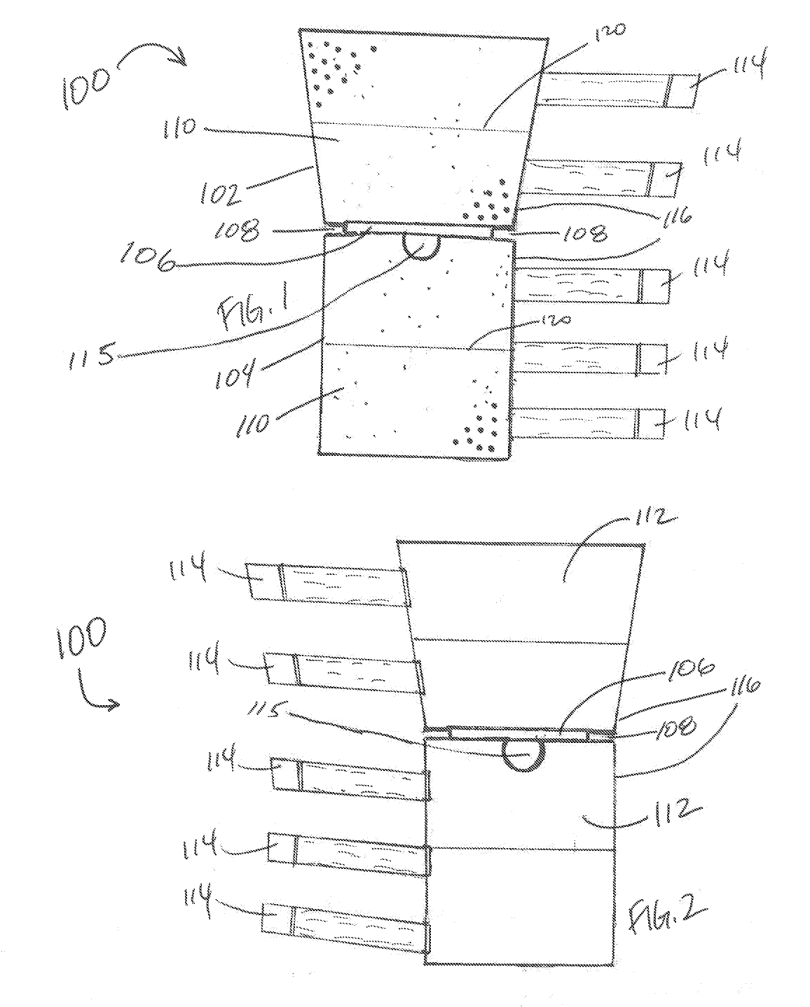

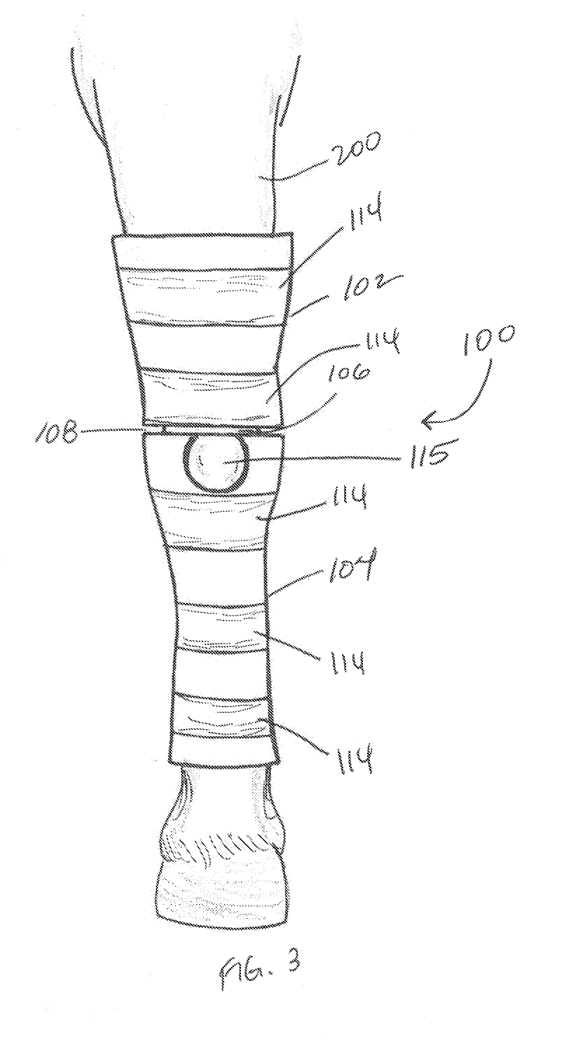

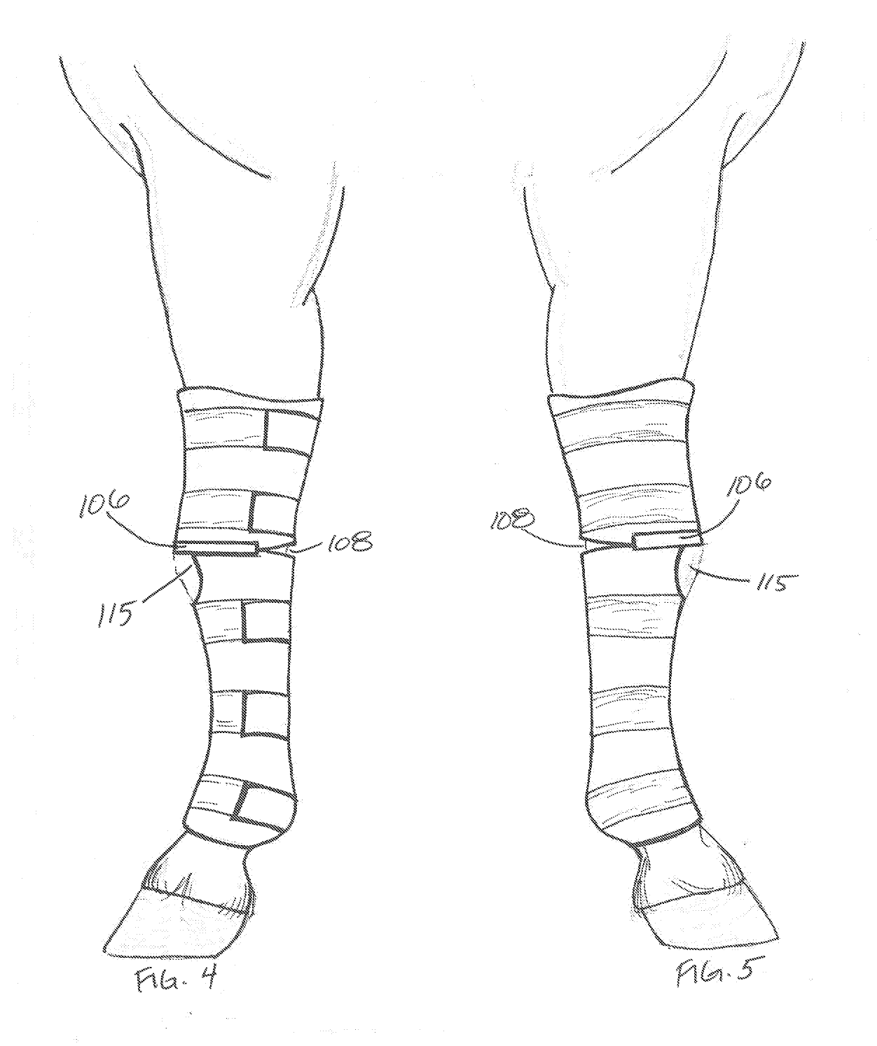

FIGS. 1-5 show an aspect of an ice therapy device 100 according to the disclosure. In some aspects, the ice therapy device is configured to provide cold therapy to many different body parts such that the ice therapy device does not inhibit mobility of the body part upon which the ice therapy device is applied. The ice therapy device is configured to be easily adjusted and/or repositioned to be properly applied to the body part. The ice therapy device can also be easily adjusted and/or repositioned during cold therapy sessions without substantially interrupting the cold therapy session. The ease of adjustability of the ice therapy device allows for altering or adjusting the arrangement of the ice therapy device based on many factors, such as but not limited to, personal preference, accommodate other body parts, provide access to part of body during cold therapy session, or the like.

The ice therapy device 100 comprises at least a first section 102, at least a second section 104, at least one joint 106, and at least one seam 108, wherein the at least one joint couples the first section and second section together. The first section 102 comprises an inner surface 110 and an outer surface 112 that are coupled together to form a pouch, wherein the pouch is configured to house at least one ice cell (not shown). The second section 104 also comprises an inner surface 110 and an outer surface 112 coupled together to form a pouch (not shown) that houses at least one ice cell. Each of the first and second sections further comprise at least one strap 114, such that a respective at least one strap 114 is configured to be removably coupled to the outer surface 112 of the first and second sections, respectively. The at least one seam 108 is adjacent the at least one joint 106 and extends towards an outer edge 116 of the first and second sections. The at least one seam 108 provides a separation between the first and second sections 102, 104, such that the ice therapy device 100 allows for mobility while the ice therapy device is applied to a body part. In some aspects, the at least one seam 108 allows the first and/or second sections 102, 104 to move relative to the other section. The at least one seam 108 allows the first and/or second sections to move during cold therapy sessions, such that the positioning of the ice therapy device is not substantially shifted.

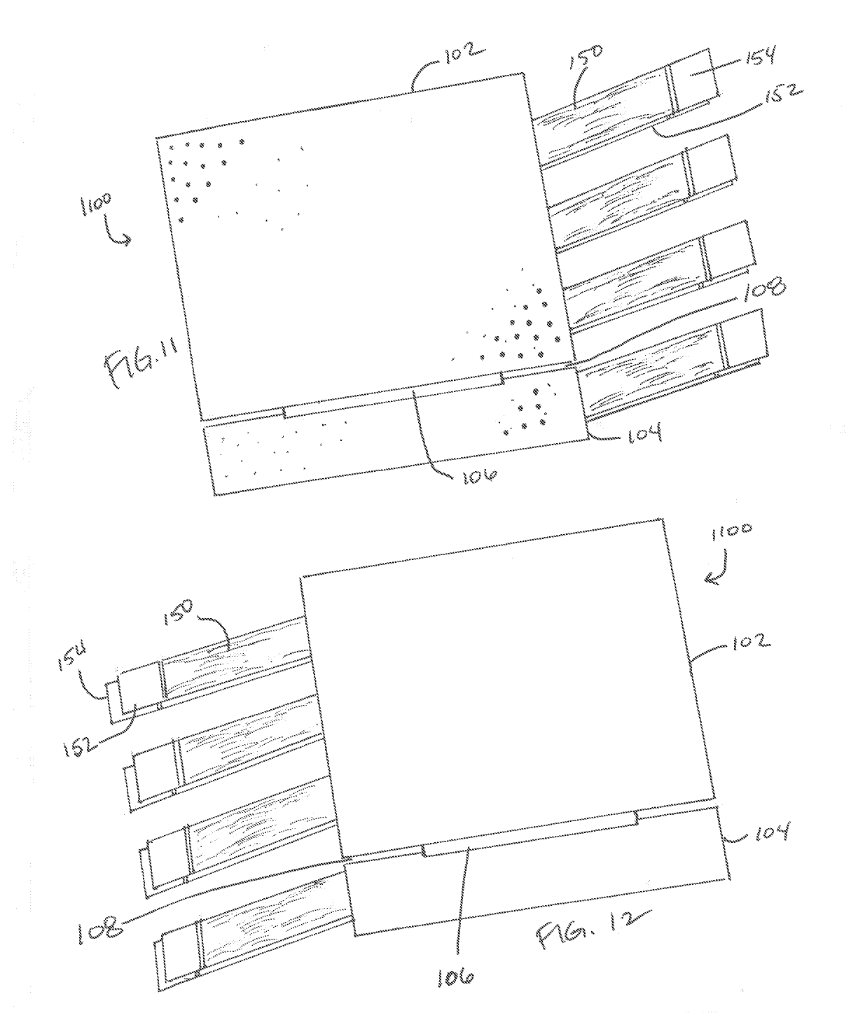

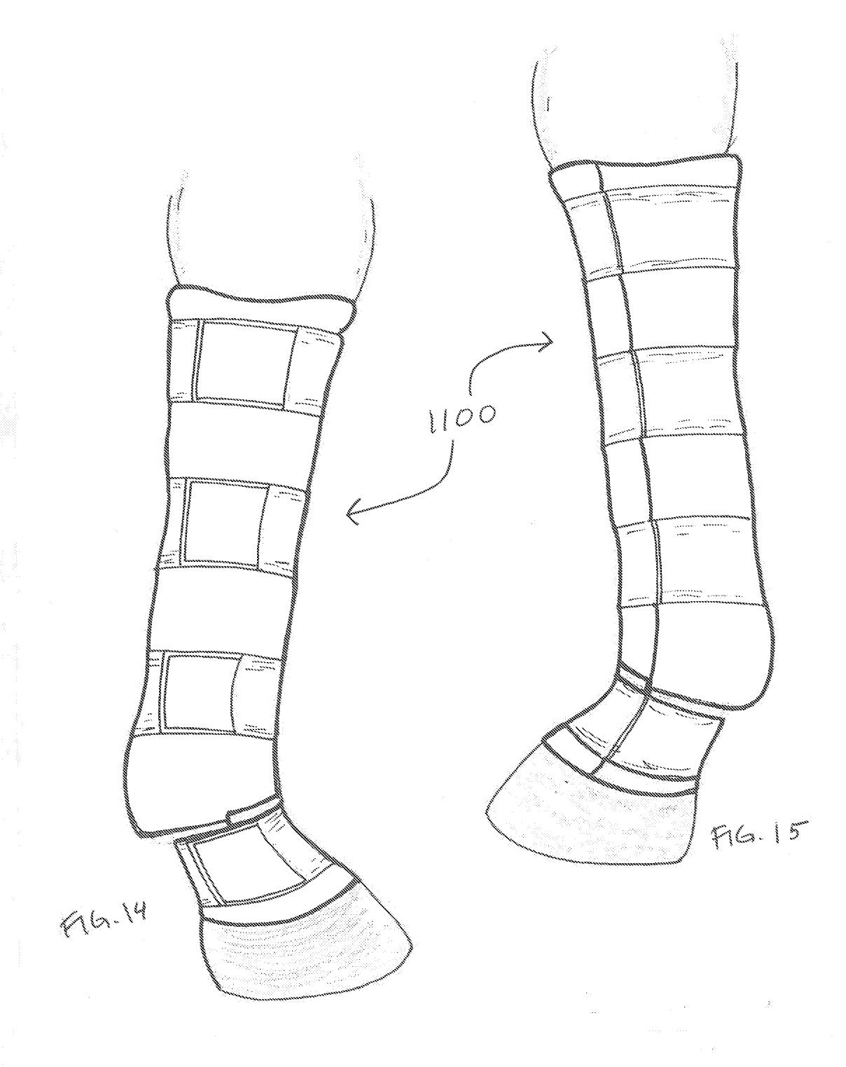

In some aspects, the straps 114 are along the same edge 116 of the first and second sections, such that a first end of the straps 114 is coupled to the first and second sections, respectively. The straps 114 can extend towards the opposite edge of the first and second sections, respectively, and can be removably coupled to the outer surface 112 of the first and second sections. In some aspects, a second end of the straps 114 can be removably coupled to the outer surface 112 of the first and second sections. The second ends of the straps can comprise an attachment device to allow the straps to be removably coupled to the first and second sections, such as but not limited to a plurality of hooks, loops, clasps, buckles, or the like, and/or a combination thereof. In some aspects, the entirety of the straps or at least part of the straps between the first and second ends can be removably coupled to the outer surface 112. In some aspects, the outer surface 112 of the first and second sections can be comprised of material having a plurality of loops, while the straps 114 can have a plurality of hooks such that the plurality of hooks are received by the plurality of loops in order to removably couple the straps to the outer surface. In some aspects, the ice therapy device can comprise a plurality of paired or double straps 150, as shown in FIGS. 11-15, wherein a first strap 152 can wrap around a front portion of the ice therapy device and a second strap 154 can wrap around a behind portion of the ice therapy device. In such an aspect, the double straps 150 can wrap around the ice therapy device and the body part within the ice therapy device to secure the ice therapy device to the body part. In some aspects, the double straps 150 can comprise a plurality of hooks and/or loops such that the double straps 150 are removably coupled to each other and/or the outer surface of the ice therapy device to secure the ice therapy device onto the body part. Additionally, the double straps 150 and/or straps 114 can cause the ice therapy device to compress onto the body part, such that the ice therapy device can provide compression therapy. In some aspects, the straps 114, 150 can adhere on substantially any area of the outer surface which allows the ice therapy device to be adjusted for differently shaped body parts, such that the ice therapy device can substantially conform to the shape of the received body parts. The first section and the second section can each independently conform to the shape of the body part received by the respective section, which allows for the sections to accommodate and/or conform to various shaped body parts. Also, the straps 114, 150 can also be adjusted to increase and/or decrease the amount of compression force being applied by the ice therapy device. The straps 114, 150 also make it easy to apply the ice therapy device so that the straps do not apply pressure to sensitive and/or injured areas. In the aspect of FIGS. 1-5, the straps 114 are along the same edge of the first and second sections, but the disclosure is not intended to be limited to such configuration. In some aspect, the straps can be located anywhere on the device, such as opposing edges, adjacent edges, or on the outer surface. In some aspects, the straps have the similar dimensions, while in some aspects, the straps have different dimensions. In aspects with double straps 150, the straps can have the same or different dimensions. The straps of any of the aspects can be comprised of elastic material and/or other lightweight material. The straps can comprise many different types of connectors to removably couple the straps to the ice therapy device, and is not intended to be limited to hook and loop type connectors. In some aspects, the straps can comprise a buckle, clips, clasps, latches, notches, the like, and/or a combination thereof, wherein the ice therapy device comprises a corresponding mechanism to engage with the straps in order to removably couple the straps.

The at least one joint 106 is configured to couple the first section 102 and the second section 104 together. The at least one joint can be comprised of robust material that supports the weight of the first and/or second sections, such that the ice therapy device does not rupture and/or break at the joint 106. In some aspects, the joint 106 can be comprised of webbing material or a ribbon material that provides structural support for the ice therapy device about the joint. The joint couples the first and second sections together such that the first and second sections are substantially hingedly coupled to each other. The first and second sections being hingedly coupled allows the ice therapy device to move such that the body part wrapped within the ice therapy device has mobility such that the body part wrapped within the ice therapy device does not experience limited and/or restricted movement. Conventional ice therapy devices for animals, such as horses, require the body part such as the leg of the horse to remain stationary while undergoing an ice therapy treatment using the convention device. Conventional ice therapy devices could cause harm to the horse leg if the horse moves during the ice therapy treatment. Also, the conventional ice therapy device could slip, fall, and/or become displaced if the horse moves, such that the body part of the horse that requires treatment is not actually receiving the treatment. At least one advantage of the ice therapy device is that the horse or user is able to move while wearing the ice therapy device and does not need to be stationary during the ice therapy treatment.

In some aspects, the joint 106 can be interposed between the first and second sections in order to couple the first and second sections together. The joint can extend between opposing edges of the first and second sections, such that the joint is coupled to each of the first and second sections. The joint can be arranged in many different configurations and is not intended to be limited to the aspects disclosed herein. In some aspects, the first and second sections can be coupled to each other, and the joint can be coupled to the first and second sections at their coupling point or where the first and second sections overlap, such that one of the first or second section is interposed between the joint and the other of the first or second section. In some aspects, the joint can be comprised of a single joint that couples the first and second sections together. In some aspects, the joint can be comprised of a plurality of joints, wherein each of the plurality of joints couples part of the first and second sections together such that the plurality of joints as a whole couple the first and second sections together. In some aspects, the joint is integral with the first and second sections wherein the joint is affixed to the first and second sections. In some aspects, the positioning of the ice therapy device can be adjusted such that the joint can accommodate the body part, as desired, for a proper fit. In some aspects, the joint is on the outer surface of at least one of the first or second sections. In some aspects, the joint is on the outer surface of both the first and second sections, while in some aspects the joint is on the outer surface of the first section, while is yet some aspects the joint is on the outer surface of the second section. The joint can be on the inner surface of at least one of the first or second sections. In some aspects, the joint can be on the inner surface of the first section and on the inner surface of the second section. In some aspects, the joint can be on the inner surface of the first section and on the outer surface of the second section. In yet some aspects, the ice therapy device can comprise a joint on the outer surface of the ice therapy device and another joint on the inner surface of the ice therapy device. The joint can be configured in many different configurations to couple the first and second sections together and is not intended to be limited to the aspects disclosed herein.

Proximate the joint 106 is the at least one seam 108. The at least one seam 108 is adjacent the at least one joint 106 and extends towards an outer edge 116 of the first and second sections. The at least one seam can extend from the end of the at least one joint to the outer edge 116. The at least one seam 108 provides a separation between the first and second sections 102, 104, such that the ice therapy device 100 allows for mobility while the ice therapy device is applied to a body part. In some aspects, the at least one seam 108 allows the first and/or second sections 102, 104 to move relative to the other section. The at least one seam 108 allows the first and/or second sections to move during cold therapy sessions, such that the positioning of the ice therapy device is not substantially shifted.

The at least one seam allows the first and second sections to wrap around a body part without impacting the positioning of the opposing section. For example, as shown in FIG. 3, the first section 102 can be wrapped around an upper part of a horse's leg 200 first, and once secured, the second section 104 can be wrapped around a lower part of the horse's leg 200. The seam 108 separates the first and second sections such that each section can be applied and/or adjusted individually and independent of the other section. The seam allows the ice therapy device to be wrapped and/or applied in sections instead of having to apply and/or wrap both sections of the ice therapy device at the same time. As shown in FIGS. 3-5, the ice therapy device is applied onto the horse's leg 200, and shows that the first section is wrapped and secured around a first part of the leg, and then the second section is wrapped and secured around the second part of the leg. The seam separates the first and second sections thereby allowing the sections to be wrapped to the leg individually and not at the same time. At least one advantage of wrapping the sections individually is that each section can be properly wrapped around the body part before wrapping the other section around the body part. The seam provides for ease of applying the ice therapy device. Conventional devices would require that both sections be applied at the same time which could result in improper application of the device or misalignment, and adjusting these conventional devices could require complete removal of the device, which could be cumbersome. The disclosure overcomes this issue because the sections of the ice therapy device are separated by the seam, and thereby allows for individual application and/or adjustment of each section.

The seam can also be arranged to be positioned proximate a body joint of a body part when the ice therapy device is applied to the body part. The seam can allow the body joint the freedom to bend while the ice therapy device is wrapped around the body part. With reference again to FIG. 1-5, the ice therapy device 100 is wrapped around the horse's front leg 200 and positioned on the horse's leg such that the seams are proximate the knee. The seams being proximate and/or positioned near the knee allows the knee to easily bend while the ice therapy device is wrapped around the leg. Because the seam provides a separation between the first and second sections, the sections do not substantially interfere with each other while the knee bends. At least one advantage of the disclosure is that an animal, such as a horse, or a person having the ice therapy device wrapped around their leg has the freedom to walk and/or bend their knee during the cold therapy session, due in part because the seam allows the first and second sections of the ice therapy device to move freely. The ice therapy device can be configured such that the seam can be positioned proximate other joints of a horse's leg and is not intended to be limited to being proximate the knee, and can also be wrapped around either the front and/or back legs. In some aspects, the ice therapy device can be wrapped around other parts of the horse's leg such that the joint and/or seam of the ice therapy device can be proximate other joints of the horse's leg, such as but not limited to, the hock, fetlock, pastern, or the like, and/or a combination thereof. The ice therapy device can also be wrapped around other body parts of a person, such as but not limited to the arms, legs, torso, and/or neck.

Proximate the joint 106 is the at least one opening 115. The at least one opening 115 can accommodate a portion of a body part wrapped within the ice therapy device 100 to further assist in allowing mobility while wearing the device 100. For example, as shown in FIGS. 3-5, the device 100 is on the horse's leg 200 and the opening 115 is positioned to be proximate the knee of the horse's leg. The opening 115 being aligned with the knee of the horse's leg allows the knee to be uncovered by the device 100 such that the device 100 does not exert a force onto the knee and/or part of the horse's leg proximate the opening 115, when the knee is bent. The opening 115 accommodates the bending of the knee so as to prevent exerting a force onto the knee and/or leg while wearing the device 100. The opening 115 further assists in providing mobility while wearing the device 100, such that the wearer can walk and/or be mobile while wearing the device. The opening 115 reduces and/or minimizes the possibility of the device 100 shifting while walking and/or bending the knee, and thereby assists in maintaining the position of the device on the horse's leg. The opening 115 of FIGS. 1-5 is shown as being within the second section 104. However, the opening 115 can be located in other sections and is not intended to being limited to being within the second section. In some aspects, the opening 115 can be within the first section 102, the second section 104, a different section, and/or a combination thereof. In yet some aspects, the ice therapy device can comprise one or more openings 115.

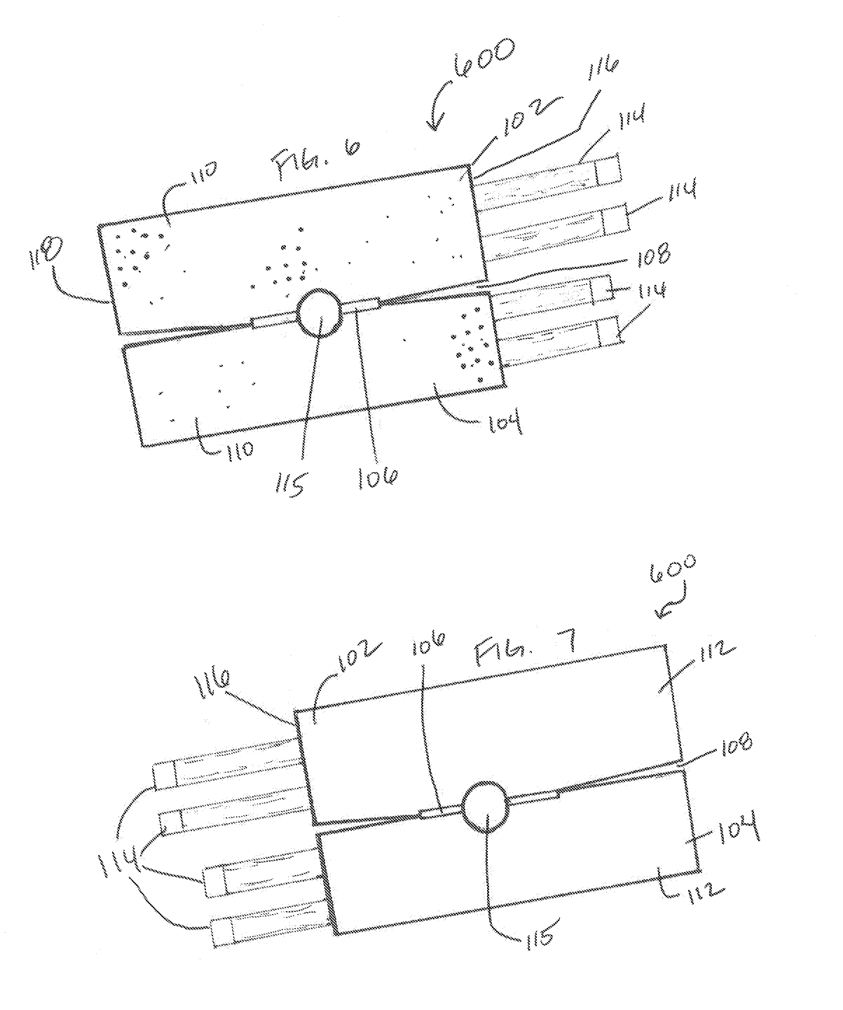

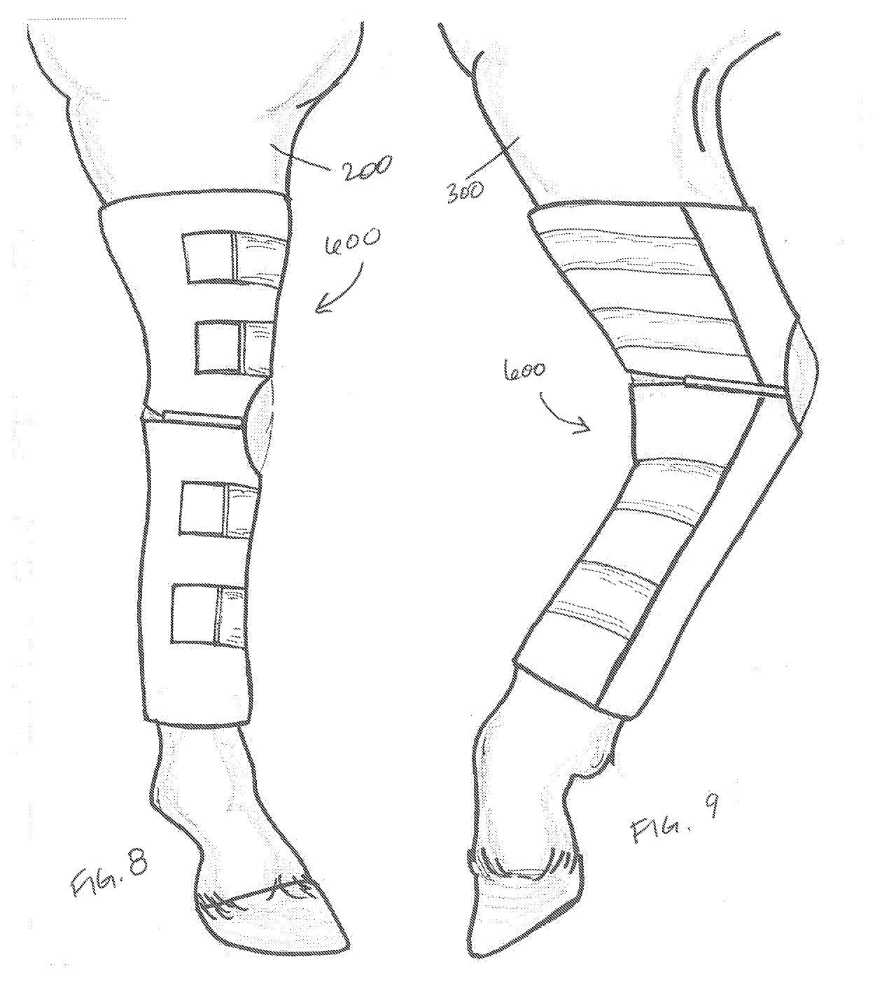

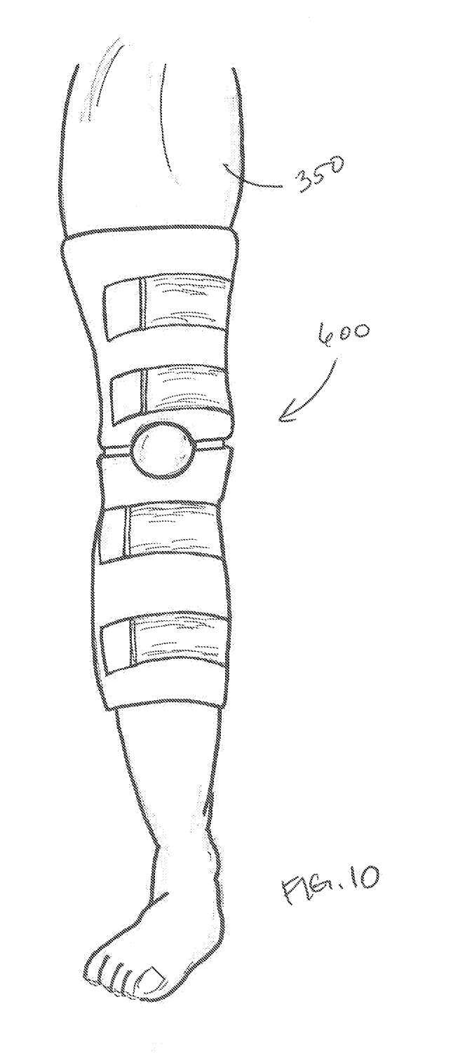

FIGS. 6-10 show an aspect of an ice therapy device 600 according to the disclosure. The ice therapy device 600 is configured in a manner similar to the ice therapy device 100 discussed above. For the same or similar features, the same reference numbers will be used throughout the disclosure herein. The ice therapy device 600 comprises a first section 102, a second section 104, at least one joint 106 coupling the first and second sections together, and at least one seam 108 proximate the joint, wherein the first and second sections comprise an inner surface 110 and an outer surface 112 to form a pouch to house at least one ice cell (not shown). The first and second sections also comprise straps 114, but can also comprise the double straps 150, as discussed above. The ice therapy device 600 further comprises an opening 115 proximate the joint 106. In the aspect of FIGS. 6-10, the opening 115 is within part of the first section 102 and within part of the second section 104, wherein a first joint 106 extends from the opening 115 toward the outer edge 116, and a second joint 106 extends from the opening 115 toward an opposing outer edge 118. The opening 115 of the ice therapy device 600 is configured to be positioned to accommodate a joint of the user that is wearing the ice therapy device 600. FIG. 8 shows an example of the ice therapy device 600 on a horse's front leg 200, while FIG. 9 shows an example of the ice therapy device 600 on a horse's rear leg 300. The opening 115 allows the horse's leg 200, 300 to bend and allow for mobility while wearing the ice therapy device, in a manner similar as discussed above.

The disclosure is not intended to be limited to the aspects disclosed herein. In some aspects, the ice therapy device is wrapped around different body parts such that the seam is positioned proximate to different body joints. The ice therapy device can also be worn by a human. For example, FIG. 10 shows an example of the ice therapy device 600 on a human leg 350. The device 600 allows the leg 350 to bend while wearing the device 600 similarly as discussed above.

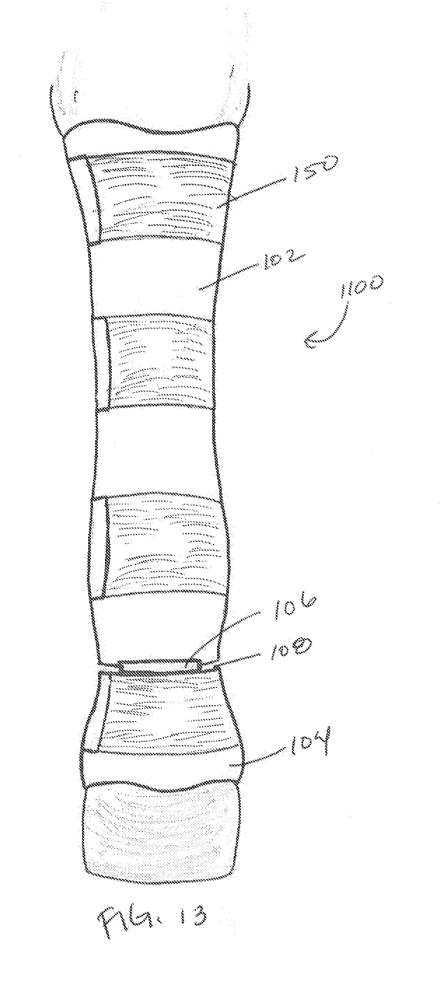

In some aspects, the first section 102 and the second section 104 are similarly shaped, as shown in FIGS. 6-10. However, in some aspects, the first and second sections are shaped differently, as shown in FIGS. 11-15. FIGS. 11-15 show an aspect of an ice therapy device 1100 according to the disclosure. The ice therapy device 1100 comprises features similar to the devices 100, 600. In the aspect of the device 1100 of FIGS. 11-15, the first section 102 of the ice therapy device 1100 is bigger than the second section 104. However, in some aspects, the device 1100 can be arranged such that the first section 102 and the second section 104 can be the same size or different sizes. Additionally, the ice therapy device 1100 shown in FIGS. 11-15 does not comprise an opening 115. However, in some aspects, the ice therapy device 1100 can comprise one or more openings 115, in a manner as discussed above. The ice therapy device 1100 can be configured to be wrapped around the lower portion of the horse's leg 200, such as but not limited to, the cannon, the fetlock, and/or the pastern. The joint 106 of the device 1100 can be positioned proximate the fetlock joint of the horse's leg 200, such that the first section 102 covers the cannon and the second section 104 covers the pastern, as shown in FIGS. 13-15. However, the device 1100 can be wrapped around other portions of the horse's leg 200 and is not intended to be limited to the lower portion of the horse's leg. The device 1100 can also be wrapped around a person's arm or leg proximate a joint, such as but not limited to a wrist, elbow, or knee. The device 1100 can be arranged to provide compression therapy due to the double straps 150, wherein the amount of compression force applied to the horse's leg can be adjusted by adjusting the positioning of the straps 150. The device 1100 still allows for mobility when worn by the horse, such that the horse can walk and does not need to be stationary. Although the aspect of FIGS. 11-15 do not show an opening 115, some aspects of the device 1100 can comprise at least one opening 115 as discussed above.

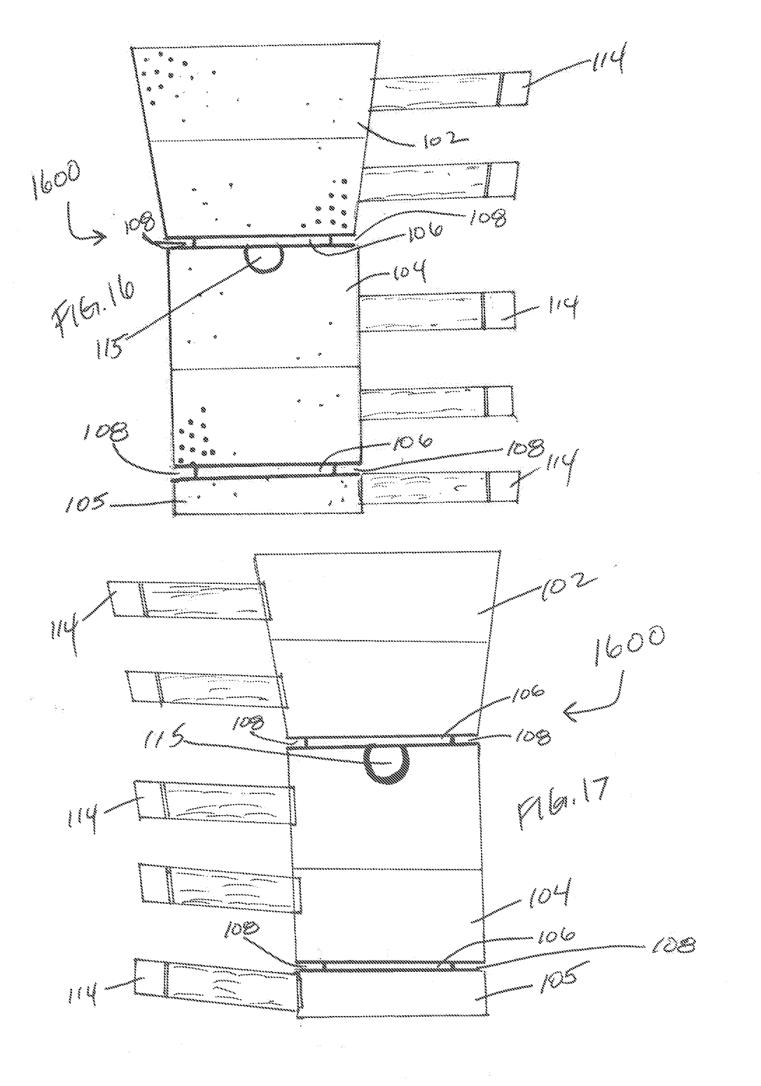

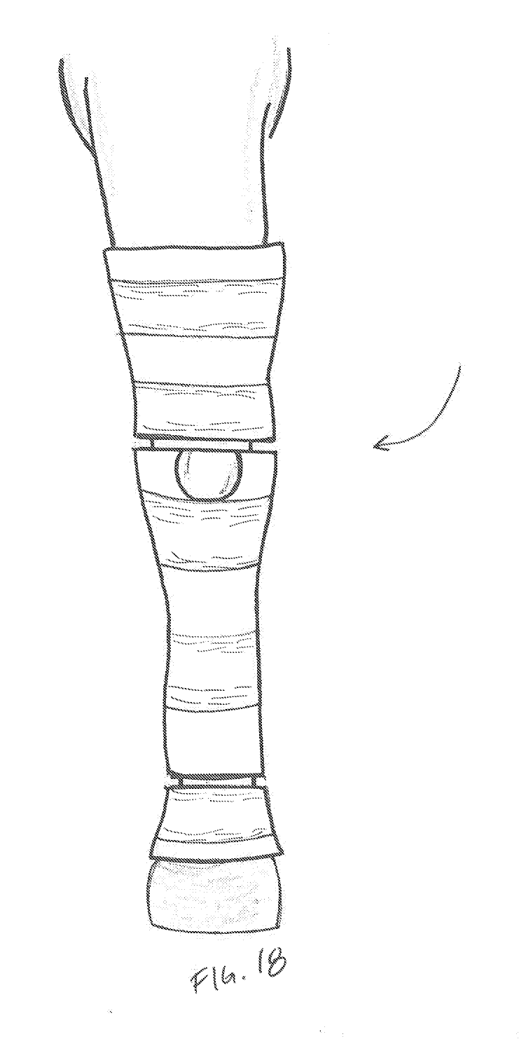

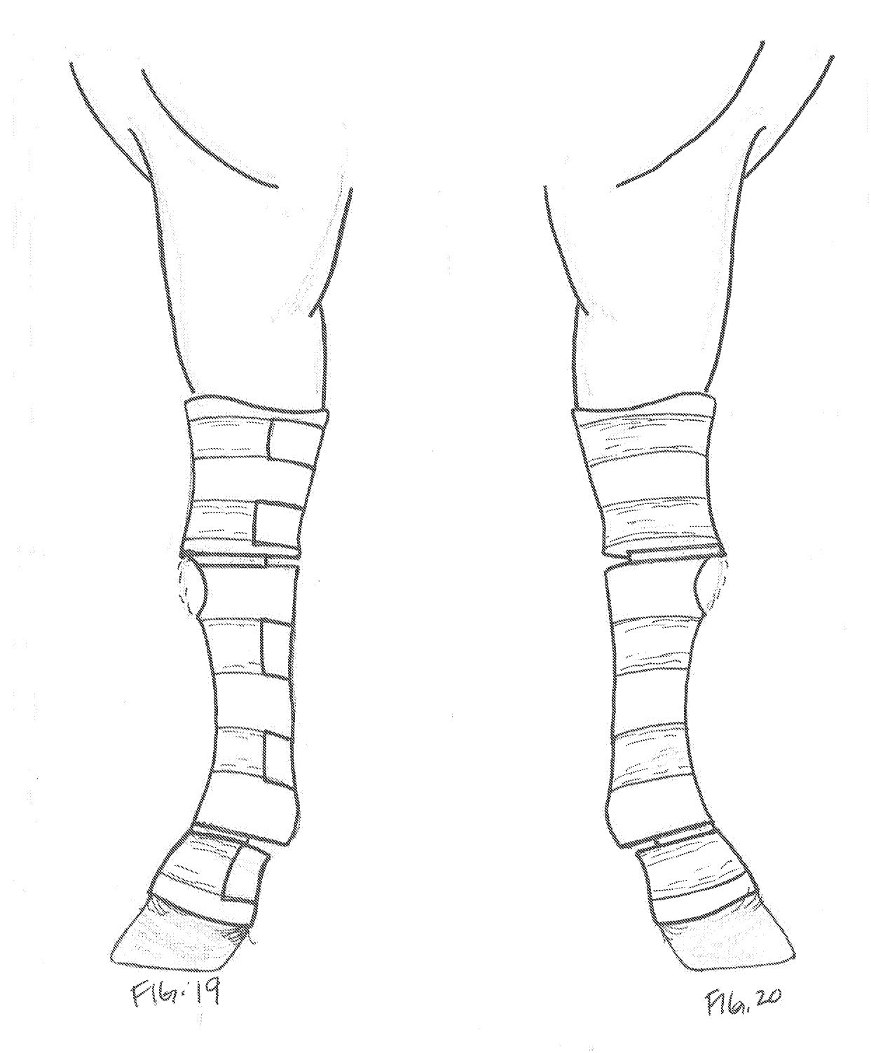

FIGS. 16-20 show an aspect of an ice therapy device 1600 according to the disclosure. The ice therapy device 1600 is configured in a manner similar to the ice therapy devices discussed above. In the aspect of FIGS. 16-20, the ice therapy device 1600 further comprises a third section 105, in addition to the first and second sections 102, 104. The ice therapy device 1600 can be configured to be wrapped around substantially all of the horse's leg 200, as shown in FIGS. 18-20. In some aspects, the first section 102 can cover the forearm or upper portion of the horse's leg above the knee, the second section 104 can cover the cannon of the horse's leg, and the third section 105 can cover the pastern of the horse's leg. The device 1600 can provide ice therapy to a substantial portion of the horse's leg 200 and yet still allow the horse to walk while wearing the device 1600. Conventional ice therapy devices require the horse's leg to be immobilized during ice therapy sessions, but the devices of the disclosure overcome the immobilization requirement of convention devices and allow the horse to walk and/or move around while wearing the device and during ice therapy sessions. The device 1600 of FIGS. 16-20 are shown with straps 114, but some aspects can comprise the double straps 150 as discussed above. The straps 114, 150 can be comprised of elastic material and can be used to apply a compression force around the body part covered by sections of the device. At least one advantage of the devices of the disclosure is that the compression force can be varied for each section based on the positioning of the straps.

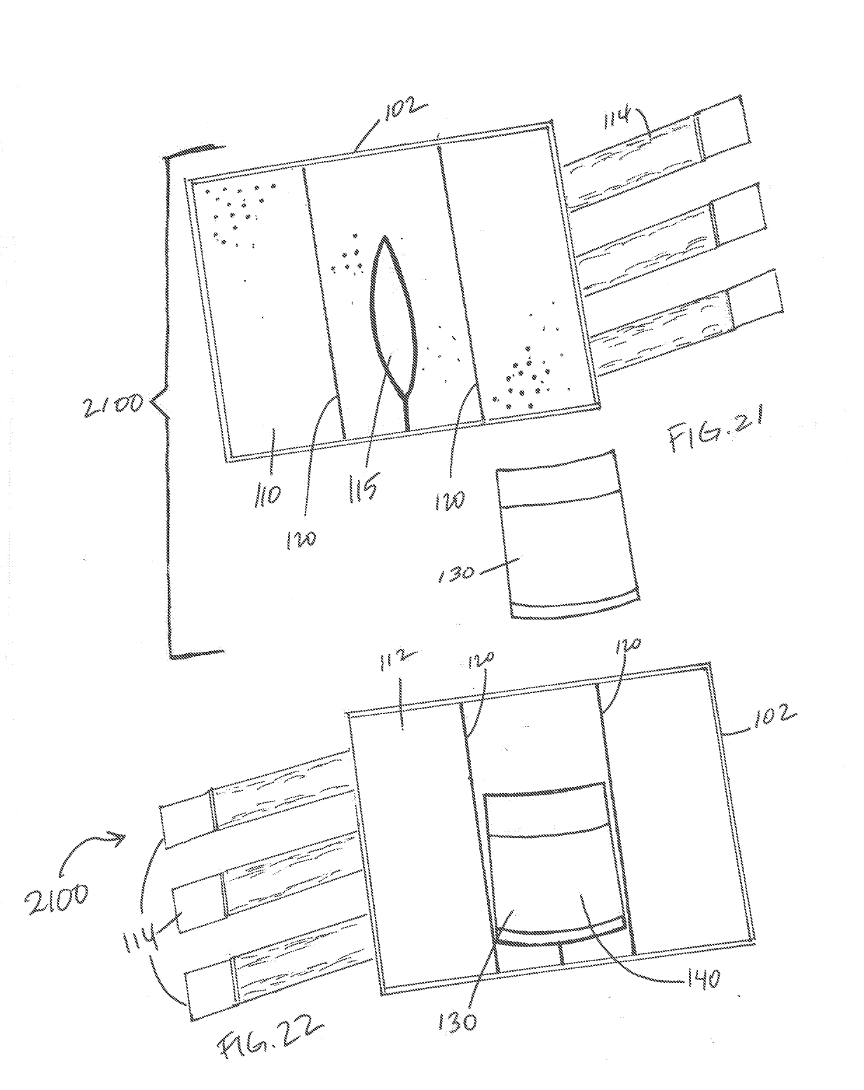

FIGS. 21-28 show an aspect of an ice therapy device 2100 according to the disclosure. The ice therapy device 2100 comprises at least a first section 102 having an inner surface 110 and an outer surface 112, a pouch interposed between the inner and outer surfaces that receives at least one ice cell, at least one strap 114 adapted to be removably coupled to the outer surface 112, an opening 115, and at least one internal seam 120. The internal seam 120 is within the first section 102 and assists in allowing the first section to easily bend around a body part of an animal or person that wears the device 2100. The internal seams 120 are configured to separate the ice cells within the first section which also assists in allowing the ice cells to easily bend. In some aspects, the internal seam 120 can form a plurality of pouches within the first section to house the ice cells. In some aspects, the ice therapy device 2100 can comprise the double straps 150, as discussed above.

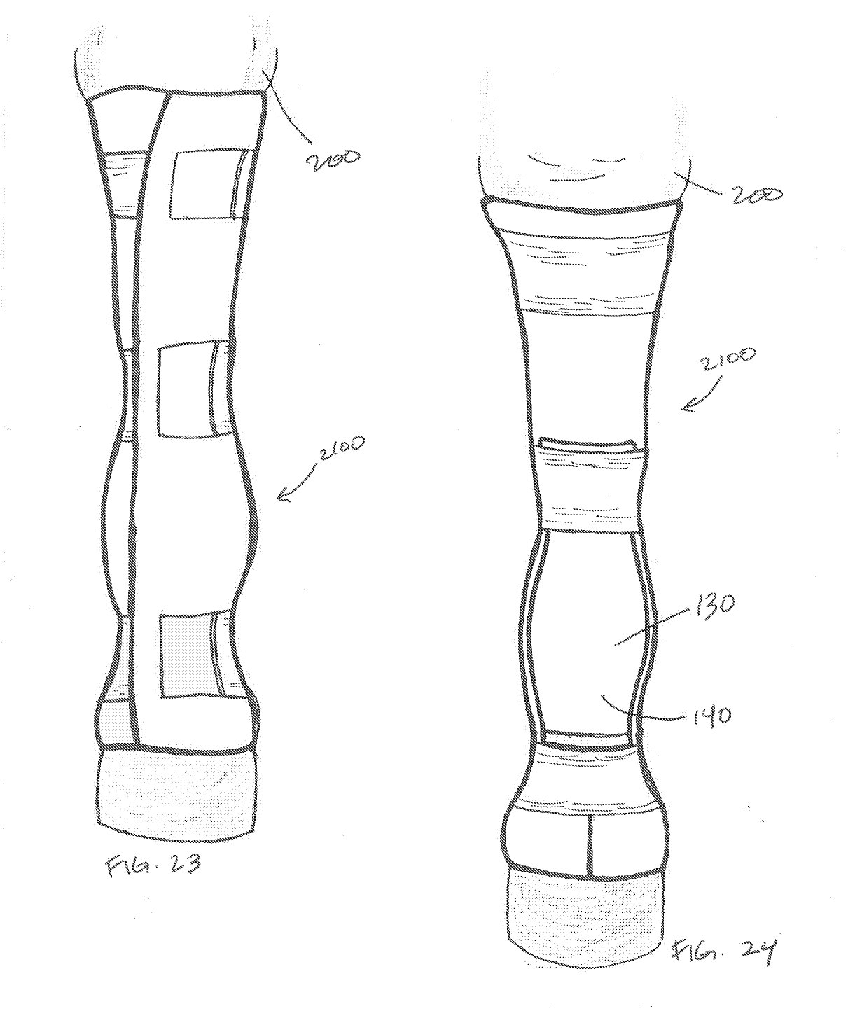

The ice therapy device 2100 further comprises a flange 130 on the outer surface 112 and proximate the opening 115. The flange 130 proximate the opening 115 can form a pocket 140. In some aspects, the pocket 140 can accommodate a body part that can extend from the ice therapy device 2100. In some aspects, the pocket 140 can be configured to receive additional ice cells and/or ice to provide ice therapy to part of the body part proximate the opening 115. In some aspects, the device 2100 can be wrapped around the horse's leg 200, as shown in FIGS. 23-26, wherein the opening 115 can be arranged proximate the proximal sesamoid bone such that the proximal sesamoid bone at least partially extends through the opening 115 such that the flange 130 covers the proximal sesamoid bone. In some aspects, ice and/or ice cells can be inserted into the pocket 140 such that ice therapy can be applied to at least part of the horse's leg 200 that extends through the opening 115. In some aspects, as shown in FIGS. 27 and 28, the ice therapy device 2100 can be wrapped around a person's arm, wherein the person's thumb extends through the opening 115 and is exposed. The device 2100 is comprised of flexible material that provides mobility to allow the wrist to bend, while the opening 115 allows the thumb to be exposed and proximate outer surface, thereby allowing the user the ability to freely use their hand. The thumb can be partially within the pocket 140 and in some aspects cold therapy can be applied by inserting ice and/or ice cells within the pocket 140.

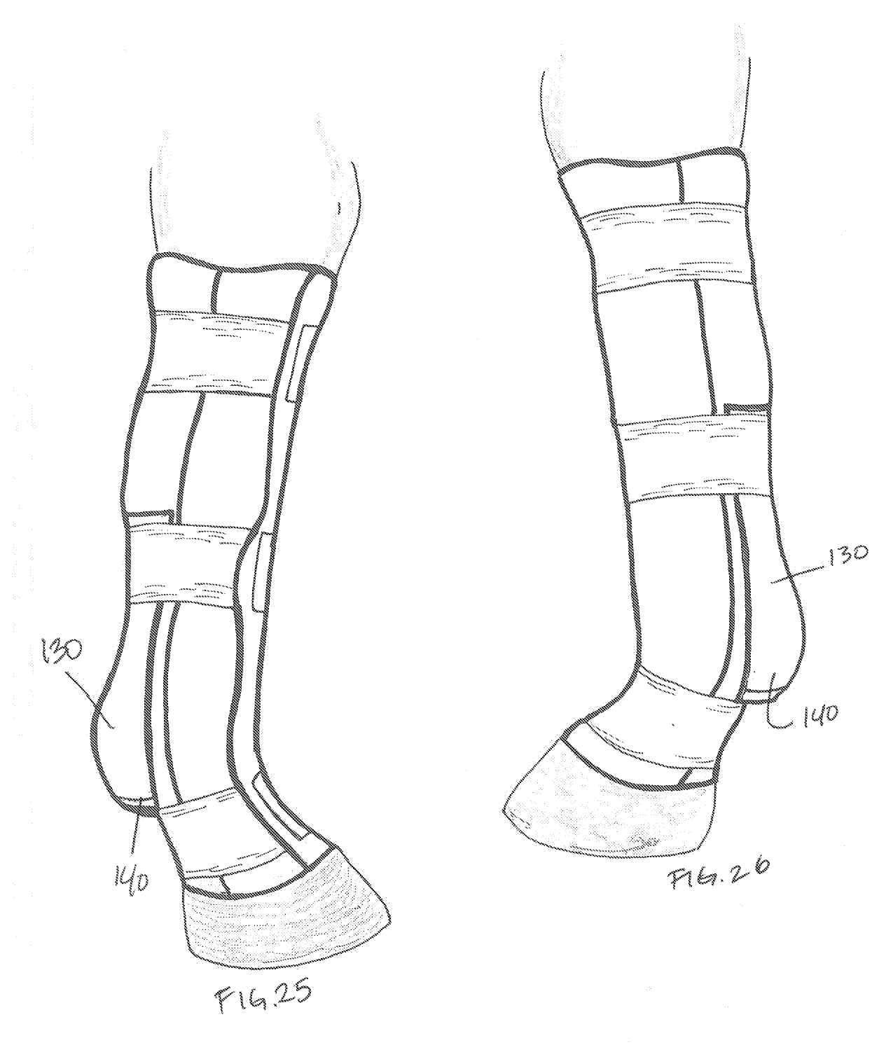

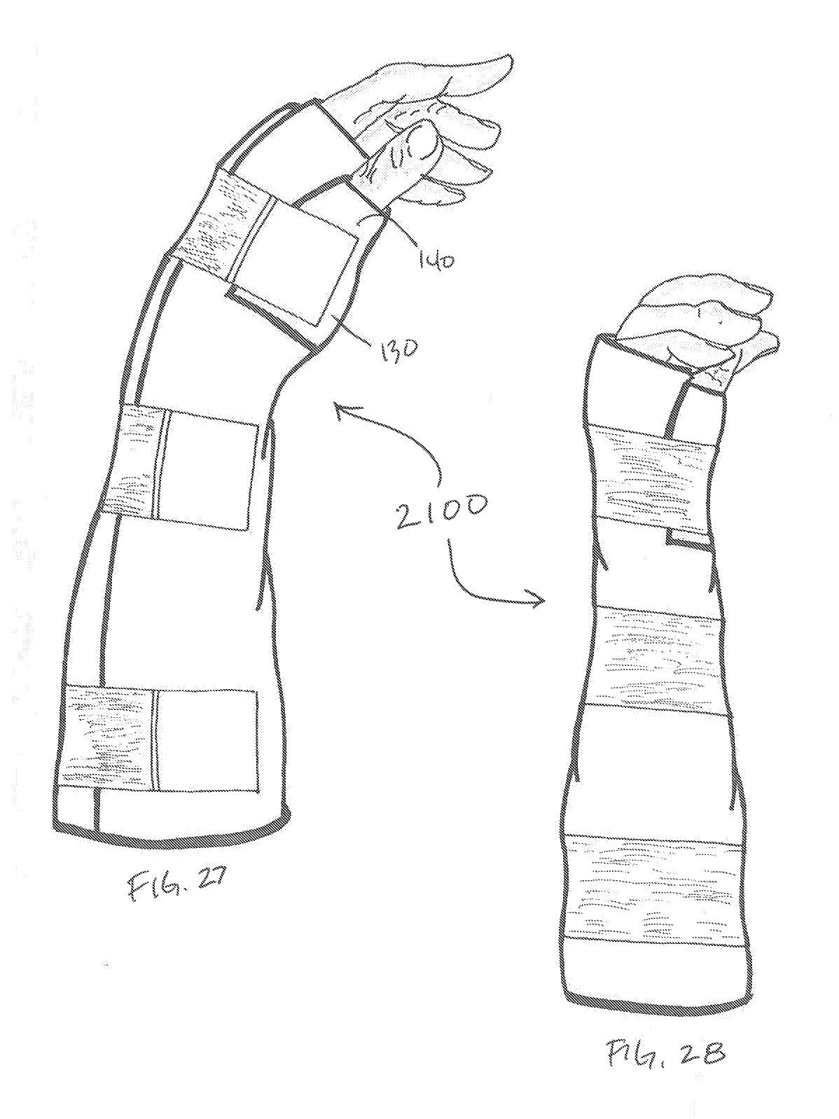

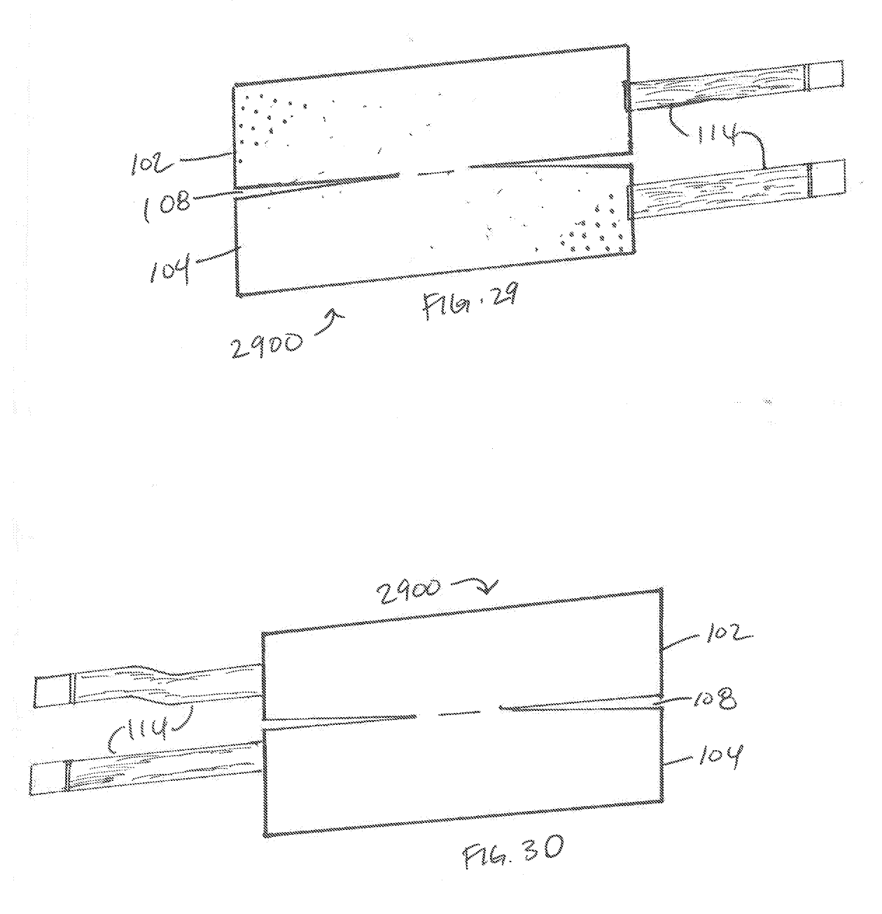

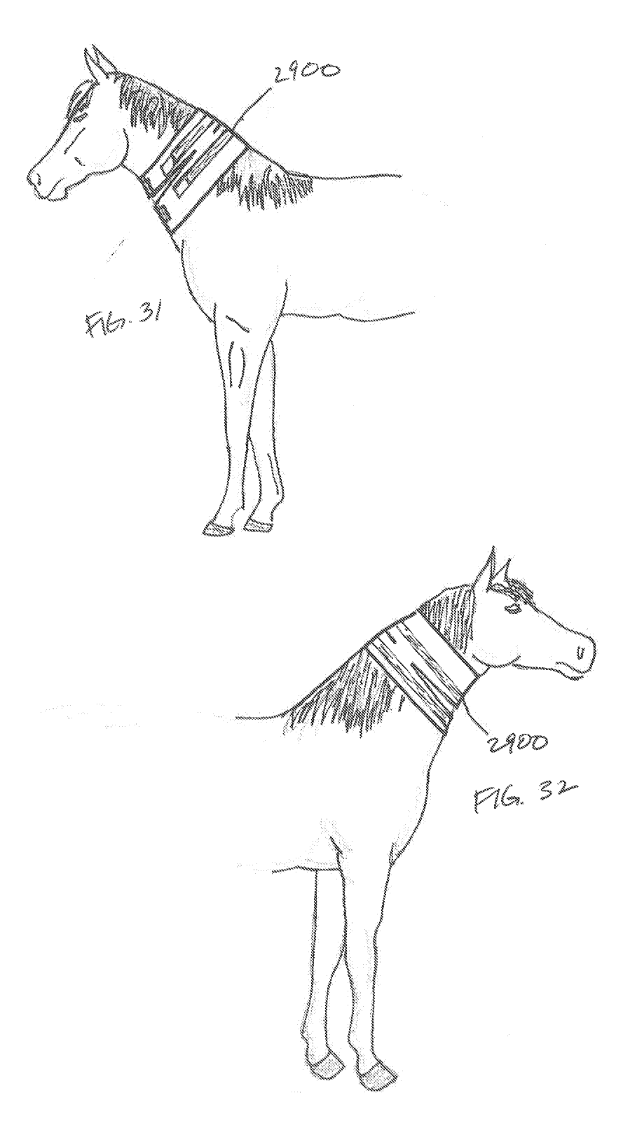

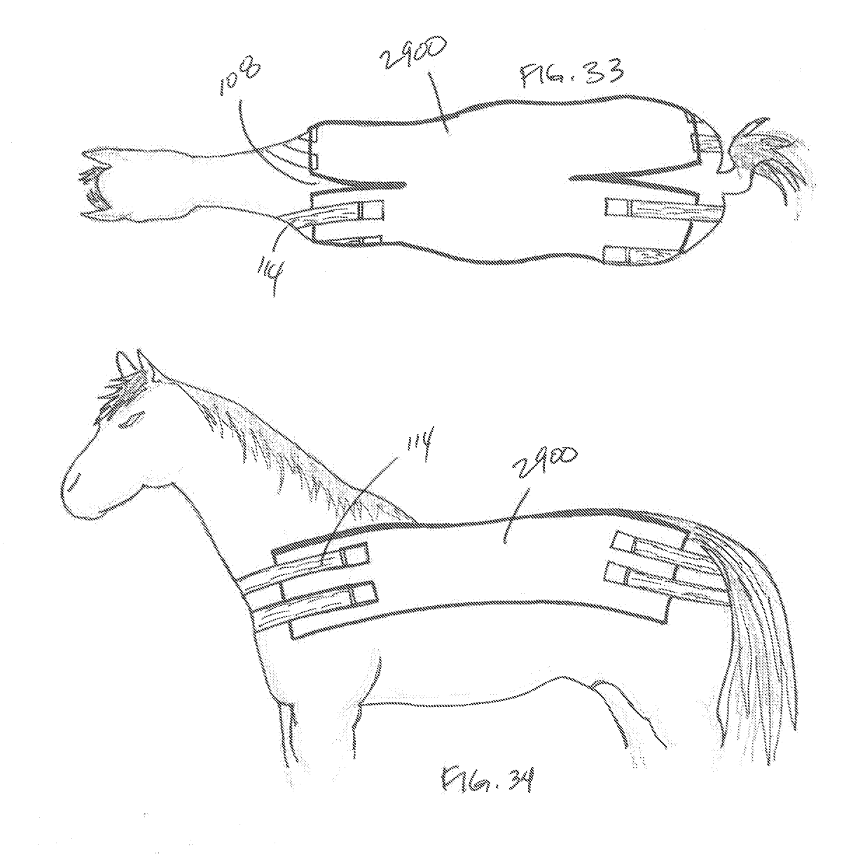

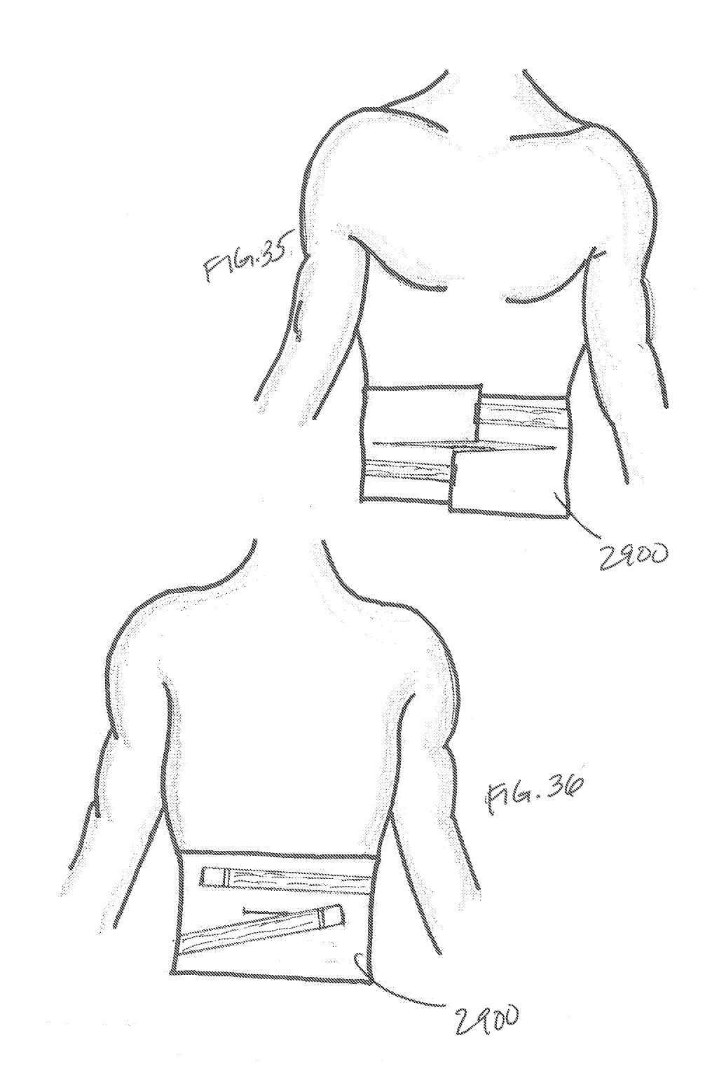

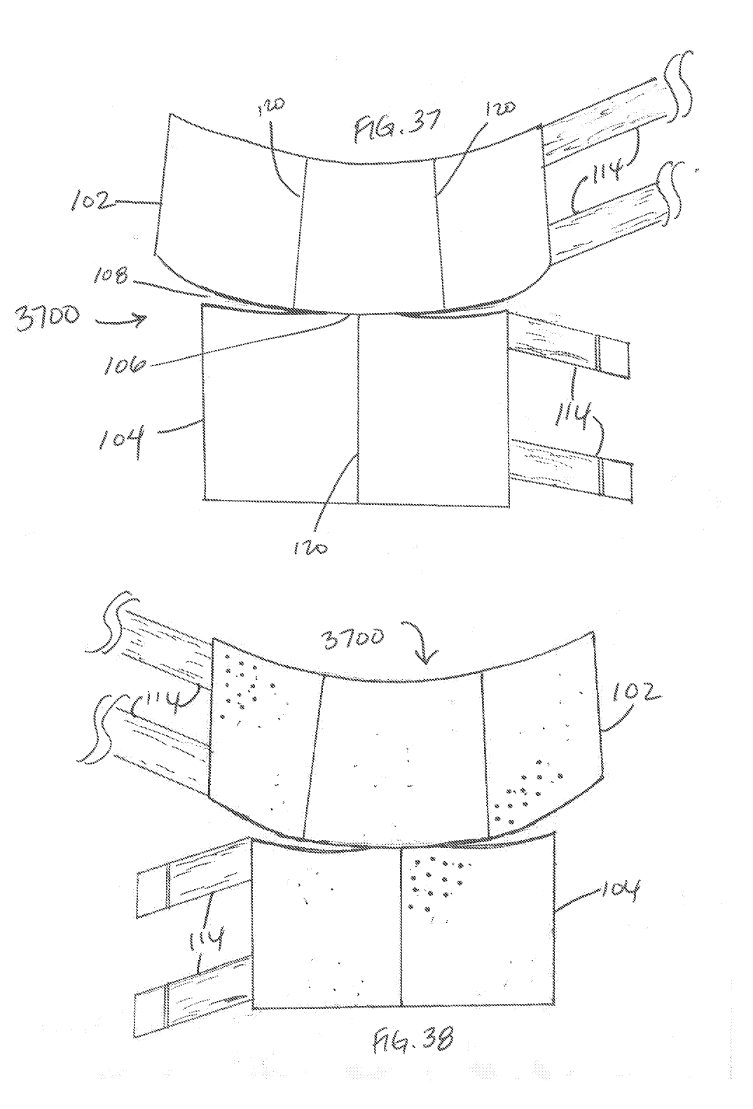

FIGS. 29-36 show an aspect of an ice therapy device 2900 according to the disclosure. The ice therapy device 2900 comprises a first section 102, a second section 104, at least one seam 108, and at least on strap 114. The ice therapy device 2900 is configured to provide ice therapy to a bigger body part, such as but not limited to a horse's neck, back, or a person's torso. As shown in FIGS. 31 and 32, the ice therapy device 2900 is wrapped around a horse's neck to provide ice therapy to the neck region of the horse. The device 2900 is comprised of flexible material and easily allows the device 2900 to be wrapped around the horse's neck. The seams 108 provide a separation between the first and second sections that allows the device 2900 to provide a better fit around the neck of the horse. The seams 108 allows the device 2900 to accommodate for the shape of the horse's neck to provide an accurate fit. The straps 114, 150 can be used to secure the device 2900 to the neck of the horse such that the device 2900 does not substantially shift or move while being worn. FIGS. 33 and 34 show an aspect of the device 2900 covering the back or loin region of the horse. In such aspect, the device 2900 can comprise straps 114 or 150 on both opposing edges of the device 2900 such that the straps at one edge can be arranged to wrap underneath neck to secure part of the device 2900, while the straps at the opposing edge can be arranged to wrap underneath the tail to secure part of the device 2900. The seams 108 can accommodate the neck and/or rear portion of the horse such that the device 2900 can substantially cover the back or loin of the horse. The straps can comprise a plurality of hooks such that the straps are removably coupled to the outer surface of the device 2900. The straps 114, 150 can be arranged in many different configurations and is not intended to be limited to the aspects disclosed herein. In some aspects, the device 2900 can comprise a plurality of grommets wherein a rope, strap, or similar structure can be inserted into the plurality of grommets such that the rope can be pulled to tighten and/or secure the device 2900 to the body part. In some aspects, the device can comprise a plurality of grooves wherein the rope, strap, or similar structure can be received within the grooves such that the rope can be pulled to tighten and/or secure the device 2900. In yet other aspects, the straps can comprise a buckle and clasp to tighten the device 2900. The straps 114, 150 in any of the aspects described herein can be configured in many different configurations and is not intended to be limited to the aspects disclosed herein. FIGS. 35 and 36 show an aspect of the device 2900 wrapped around the torso of a person, and can be arranged and/or operated in a manner as discussed in the aspect shown in FIGS. 31 and 32.

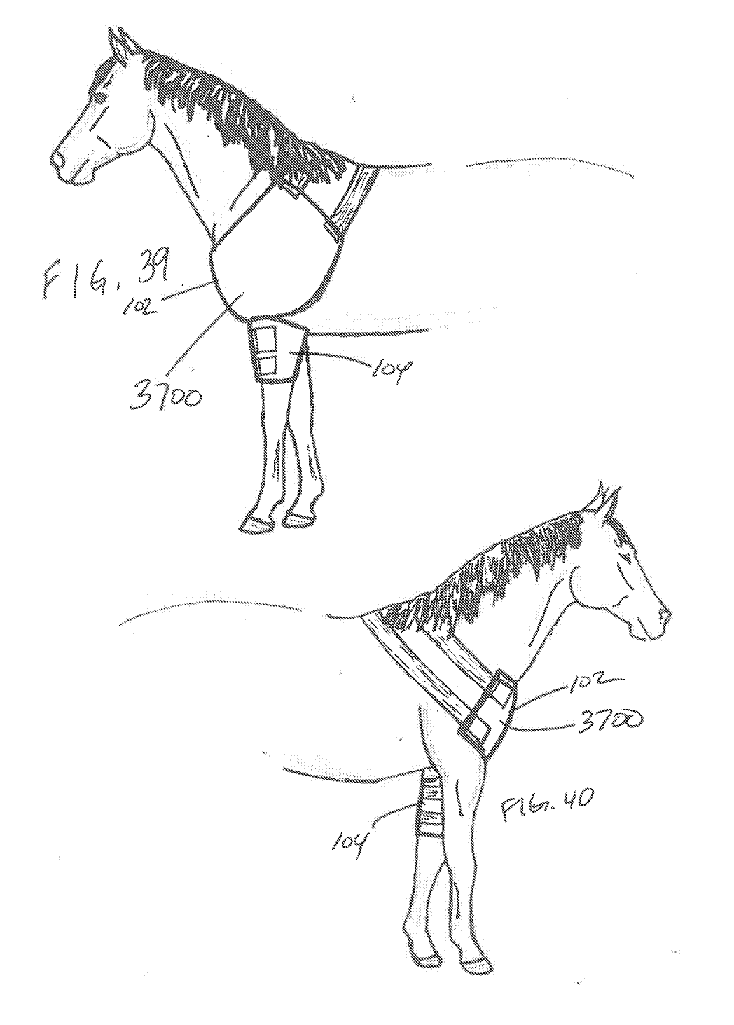

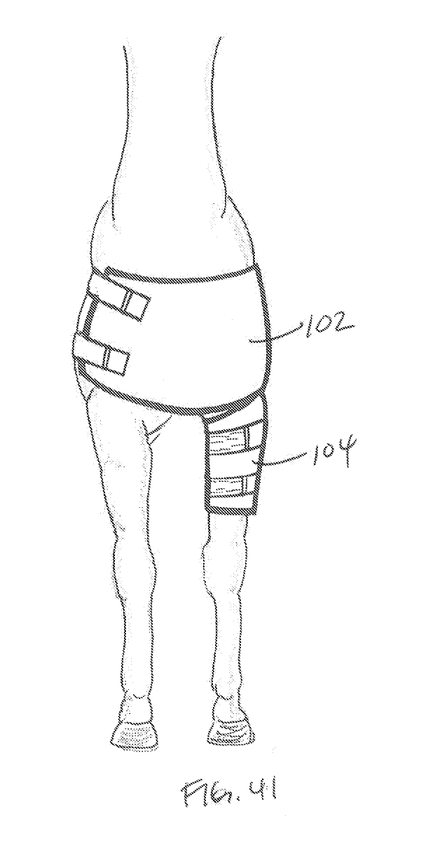

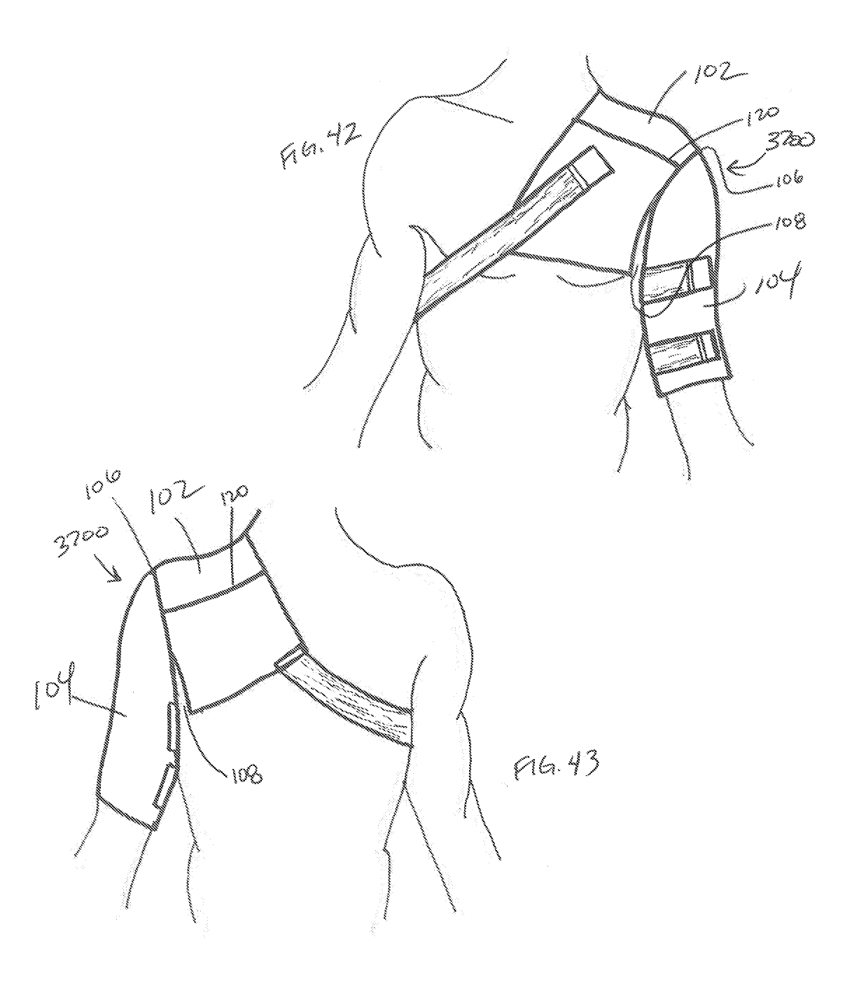

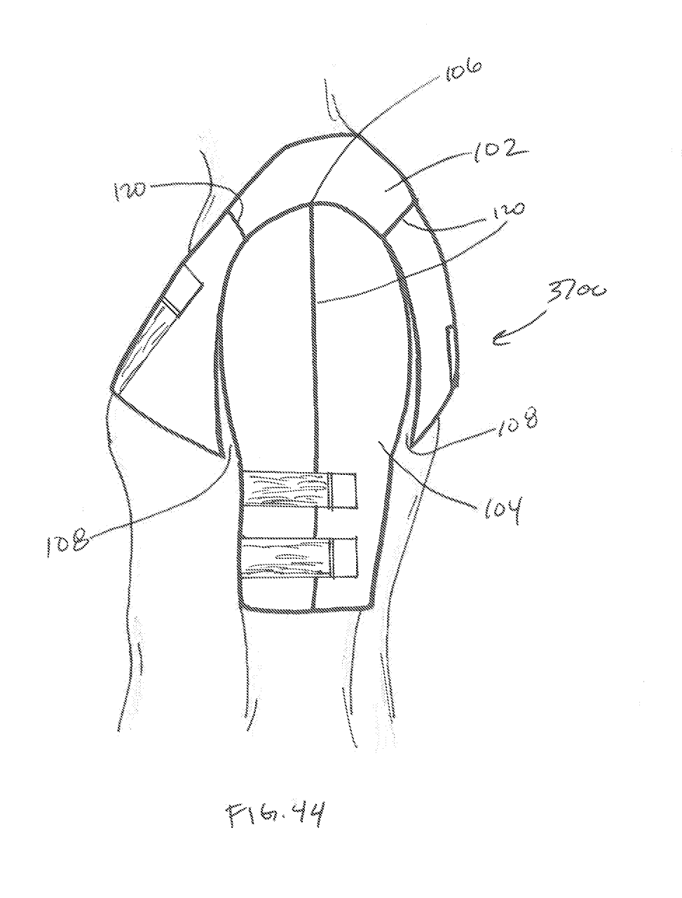

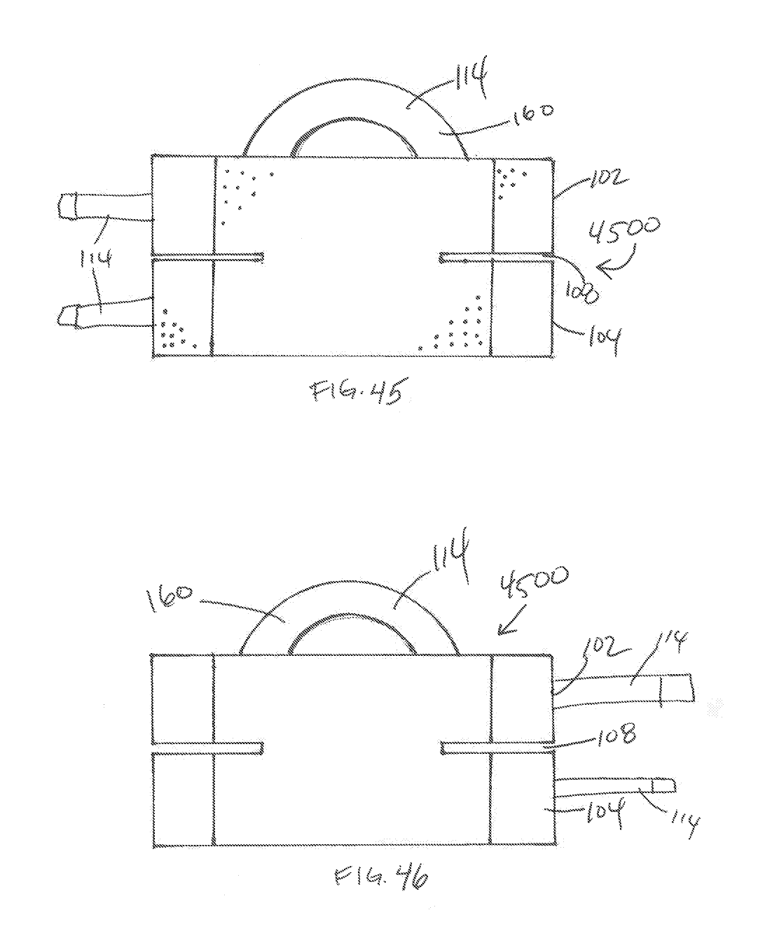

FIGS. 37-44 show an aspect of an ice therapy device 3700 according to the disclosure. The device 3700 is configured to cover multiple body parts of a horse, such as but not limited to the breast, forearm, elbow, or the like, and/or a combination thereof, as shown in FIGS. 39-41. The device 3700 can also be configured to cover multiple body part of a person, such as but not limited to, the shoulder, upper arm, chest, or the like, and/or a combination thereof, as shown in FIGS. 42-44.

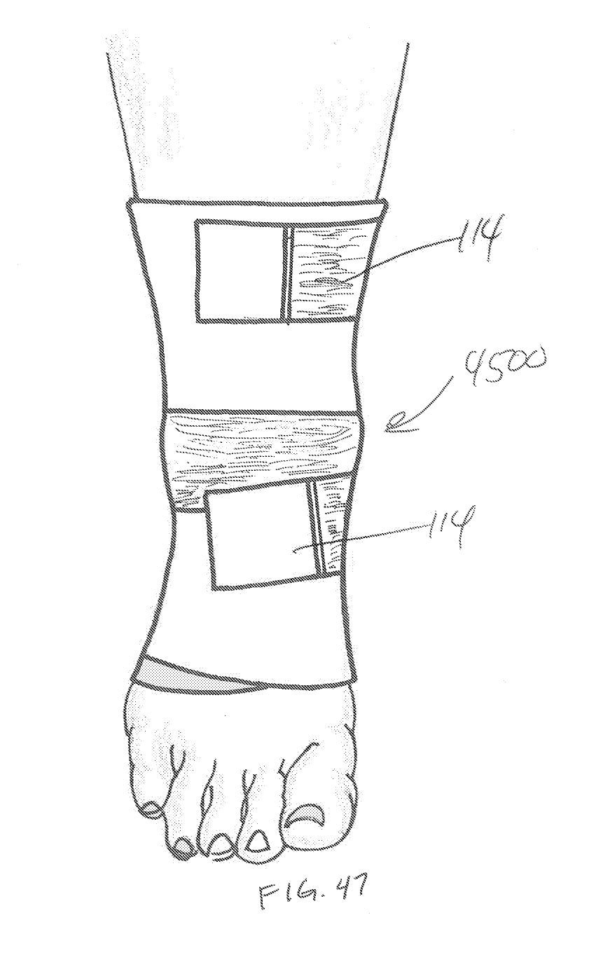

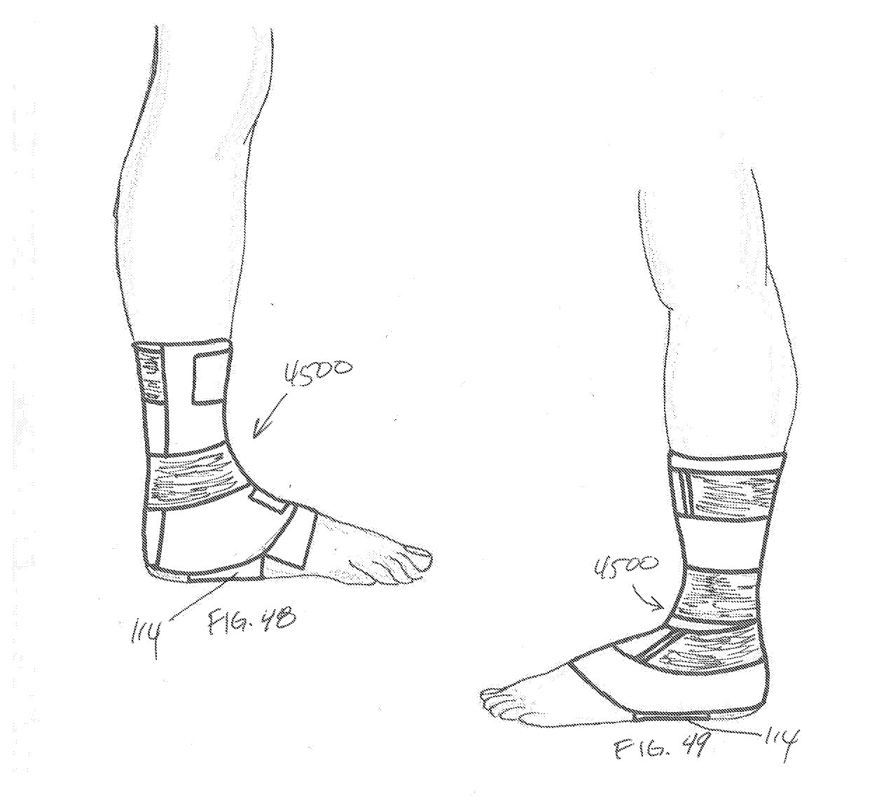

FIGS. 45-49 show an aspect of an ice therapy device 4500 according to the disclosure. The device 4500 can be configured to cover the ankle and part of the foot of a person. The device 4500 comprises a first section 102, a second section 104, a plurality of straps 114, at least one seam 108 separating the first and second sections, wherein one of the plurality of straps is coupled to the first section 102 forming a closed loop 160, wherein the closed loop is configured to wrap underneath the foot to assist in securing the device 4500 to the person. The loop 160 assists in securing the device 4500 to the person while wearing the device 4500 such that the device 4500 does not move and/or come off. The seams 108 allows the device 4500 to be wrapped around the ankle and part of the foot proximate the ankle. Each section 102, 104 comprises ice cells (not shown) within each respective section, as discussed above. The straps 114 wrap around the ankle and are removably coupled to the device 4500 to secure the device 4500 to the ankle, and allows the wearer to walk while wearing the device 4500 such that the wearer can be mobile while undergoing an ice therapy session.

The disclosure is not intended to be limited to the aspects disclosed herein. In some aspects, the ice therapy device is wrapped around different body parts such that the seam is positioned proximate to different body joints, such as but not limited to, a wrist, elbow, shoulder, ankle, hip or the like. At least one advantage of the disclosure is that the ice therapy device is not limited to being used on animals, but can also be used on humans. The ice therapy device can be configured in many different ways and is not intended to be limited to being positioned to the joints disclosed herein. In some aspects, the ice therapy device can be configured such that at least one seam can be proximate one or more joints. In some aspects, the ice therapy device can be configured such that at least one seam can be proximate any joint of a person or a horse. In yet other aspects, any of the ice therapy device disclosed herein can comprise an internal seam within at least one section such that the internal seam does not provide a separation between the at least one section. In some aspects, the first section comprises one or more internal seams such that the first section can easily bend around a body part of a person or animal. In some aspects, the second section can comprise at least one internal seam that also allows the second section to easily bend around a body part of the person or animal. The internal seams separate the ice cells within the first and/or second sections which allows them to easily bend. In some aspects, the internal seam forms a plurality of pouches within the first and/or second sections to house the ice cells.

At least another advantage of the disclosure is that the opening can reduce and/or minimize pressure applied onto the limb joint by the ice therapy device when the limb joint is bent. For example, in an aspect where the ice therapy device is wrapped around a person's leg, bending the knee joint could cause the at least one joint of the ice therapy device to exert some pressure onto the knee joint, due in part because the knee joint could be substantially covered by the at least one joint of the ice therapy device. The inclusion of the opening reduces the pressure exerted onto the knee joint when the knee is bent, because the knee joint is at least partially exposed by the opening and is not substantially covered by the at least one joint of the ice therapy device.

At least another advantage of the disclosure is that the opening can be positioned around many different body parts and is not intended to be limited to being arranged a body joint. In some aspects, the opening can be arranged proximate a body part to allow for access to the body part when the ice therapy device is wrapped around the body part. For example, the ice therapy device can be arranged such that the opening exposes any sensitive or injured areas that are not intended to have an application of cold therapy. Furthermore, the opening can provide access to the body part to insert monitors, instruments and the like to conduct measurements of the body part during the cold therapy session. The opening can be located in many different locations and is not intended to being limited to the aspects disclosed herein. In some aspects, the opening is not a central opening proximate the joint, but can be located anywhere on the ice therapy device. In some aspects, the ice therapy device comprises a plurality of openings.

Yet another advantage of the disclosure is that the device is reusable such that the ice cells can be refrozen and used repeatedly. The device can be placed in a freezer or other cold environment to allow the ice cells to freeze. Once frozen, the device can be applied to the desired body part. An advantage of the device is that the ice cells eliminate the need for ice, which reduces costs per ice therapy session and substantially eliminates water leaking from melting/melted ice, such that the device can be used in many different environments without concern for water damage due to melted ice.

Although the disclosure has been described in considerable detail with reference to certain configurations thereof, other versions are possible. The ice therapy device can be comprised of many different materials. In some aspects, the ice therapy device comprises a soft/stretch combination of exterior and interior fabrics that assist with body movement. The interior fabric allows for safe cold transfer to the body part rather than direct coldness to the skin, which protects from over freezing. The straps are arranged to accommodate various body shapes, sizes and types, and can provide compression. The ice cells can be configured to be cut fit for various ice therapy devices. In some aspects, the pouches that house the ice cells are sown shut such that the ice cells are not removable, which eliminates the need to refill the pouches with ice. The ice cells are reusable and can be refrozen after use. The ice cells can be comprised of a plurality of cell sacs filled with water, refrigerant gel, liquid or the like that can be reused to provide ice therapy. However in other aspects, the pouch can be arranged to be opened such that the ice cells can be removed, or allow for ice cells of different sizes to be used. In yet some aspects, the ice cells can be replaced with heating pads that fit within the pouch, such that the ice therapy device can provide heat therapy. The ice therapy device is universal which allows the ice therapy device to be used on humans or animals. As such, the ice therapy device is a multi-purpose device and can be used for various human and animal body parts. Therefore, the spirit and scope of the disclosure should not be limited to the versions described herein.

I claim:

1. A therapy device, comprising:
a first section configured to house at least a first ice cell;
a second section configured to house at least a second ice cell;
at least one joint interposed between the first and second sections;
at least one seam configured to provide a separation between part of the first and second sections;
each of the first and second sections comprising at least one strap, wherein the at least one strap of the first section is removably coupled to the first section, and wherein the at least one strap of the second section is removably coupled to the second section, such that each of the first and second sections are configured to receive a body part in order to apply an ice therapy treatment.

2. The therapy device of claim 1, further comprising an opening proximate the at least one joint, wherein the opening can accommodate part of the body part within the therapy device.

3. The therapy device of claim 2, wherein the opening is within at least one of the first or second sections.

4. The therapy device of claim 2, wherein the opening is within both of the first and second sections.

5. The therapy device of claim 1, the first section comprising an inner surface and an outer surface coupled together to form a first pouch, wherein the first pouch is configured to house the at least a first ice cell.

6. The therapy device of claim 5, the second section comprising an inner surface and an outer surface coupled together to form a second pouch, wherein the second pouch is configured to house the at least a second ice cell.

7. The therapy device of claim 6, wherein the first and second pouches are sealed, such that the respective at least a first and second ice cells are fixed within the first and second sections.

8. The therapy device of claim 6, wherein the first and second pouches are arranged to be opened, such that the respective at least a first and second ice cells can be removed from within the first and second sections.

9. The therapy device of claim 1, wherein each of the at least one strap of the first and second sections is comprised of a double strap, wherein a first strap of the double strap can wrap around the front of the body part and a second strap of the double strap can wrap around behind the body part such that the first and second straps are removably coupled to each other.

10. The therapy device of claim 1, further comprising a third section and a second joint, wherein the second joint is interposed between the second section and the third section.

11. The therapy device of claim 10, further comprising at least one second seam, wherein the at least one second seam provides a separation between part of the second and third sections.

12. The therapy device of claim 11, wherein the third section comprises at least one strap, wherein the at least one strap of the third section is removably coupled to the third section, such that the third section is configured to receive a third body part in order to apply the ice therapy treatment.

13. The therapy device of claim 12, wherein the third section comprises an inner surface and an outer surface coupled together to form a third pouch, wherein the third pouch is configured to house at least a third ice cell.

14. An ice therapy device, comprising:
a first section configured to house at least a first ice cell;
a second section configured to house at least a second ice cell;
first means for supporting the first section proximate the second section, in which the first means maintains a connection between the first and second sections;
at least one seam interposed between the first and second sections, wherein the at least one seam provides a separation between the first and second sections;
first attachment means coupled to the first section at a first end, in which a second end of the first attachment means is removably coupled to the first section; and
second attachment means coupled to the second section at a first end, in which a second end of the second attachment means is removably coupled to the second section.

15. The ice therapy device of claim 14, further comprising an opening proximate the first means, wherein the opening is within at least one of the first or second sections.

16. The therapy device of claim 14, wherein the first section comprises an inner surface and an outer surface coupled together to form a first pouch, wherein the at least a first ice cell is received within the first pouch, and wherein the second section comprises an inner surface and an outer surface coupled together to form a second pouch, wherein the at least a second ice cell is received within the second pouch.

17. The therapy device of claim 14, further comprising:
a third section proximate the second section;
a second means for supporting the second section proximate the third section, in which the second means maintains a connection between the second and third sections; and
at least a second seam interposed between part of the second and third sections and configured to provide a separation between the second and third sections.

18. The therapy device of claim 17, further comprising a third attachment means coupled to the second section at a first end, wherein a second end of the third attachment means is removably coupled to the third section.

19. A device sized for placement around at least one body part of an animal or human for applying a thermal treatment, the device comprising:
a first section configured to receive a first body part, the first section comprising an inner surface, an outer surface, and at least a first ice cell interposed between the inner and outer surfaces, such that that first section can substantially confirm to the shape of the first body part;
a second section proximate the first section, wherein the second section is configured to receive a second body part, the second section comprising an inner surface, an outer surface, and at least a second ice cell interposed between the inner and outer surfaces, such that that second section can substantially confirm to the shape of the second body part;
at least one joint interposed between the first and second sections, wherein the at least one joint provides structural support to maintain a connection between the first and second sections;
at least one seam proximate the at least one joint and interposed between the first and second sections, such that the at least one seam provides a separation between part of the first and second sections;
an opening proximate the at least one joint, wherein the opening is configured to receive a joint of the horse or human the connects the first and second body parts; and
each of the first and second sections comprising at least one strap, wherein the at least one strap of the first section is removably coupled to the first section, and wherein the at least one strap of the second section is removably coupled to the second section;
wherein the opening allows for the joint of horse or human to freely bend while wearing the device;
wherein the first and second straps assist to securely fasten the device to the at least one body part of the horse or human such that the horse or human has mobility during the application of the thermal treatment.
